SEQUENCE LISTING (6 sequences)

SEQ ID NO: 1 - protein, 571 aa, Human adenovirus 5
MRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 2 - protein, 207 aa, Homo sapiens
ELLPPQLKEMKSQESAAGSKLVLRCETSSEYSSLRFKWFKNGNELNRKNKPQNIKIQKKPGKSELRINKASLADSGEYMCKVISKLGNDSASANIAIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELY

SEQ ID NO: 3 - protein, 796 aa, synthetic construct
  REGION 1..796: note = Synthetic Construct
ELLPPQLKEMKSQESAAGSKLVLRCETSSEYSSLRFKWFKNGNELNRKNKPQNIKIQKKPGKSELRINKASLADSGEYMCKVISKLGNDSASANIAIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYGGSGGSRSMRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTFKKKKKKKKKK

SEQ ID NO: 4 - protein, 10 aa, synthetic construct
  REGION 1..10: note = Synthetic Construct
KKKKKKKKKK

SEQ ID NO: 5 - DNA, 30 bp, synthetic construct
  misc_feature 1..30: note = Synthetic Construct
  source 1..30: mol_type = other DNA
cgcctgagcaacgcggcgggcatccgcaag

SEQ ID NO: 6 - DNA, 30 bp, synthetic construct
  misc_feature 1..30: note = Synthetic Construct
  source 1..30: mol_type = other DNA
gcggactcgttgcgccgcccgtaggcgttc

(19) United States
(12) Patent Application Publication
(10) Pub. No.: US 2025/0255978 A1
(43) Pub. Date: Aug. 14, 2025

(54) DRUG-DELIVERY NANOPARTICLES AND TREATMENTS FOR DRUG-RESISTANT CANCER

(71) Applicant: Cedars-Sinai Medical Center, Los Angeles, CA (US)

(72) Inventor: Lali K. MEDINA-KAUWE, Porter Ranch, CA (US)

(21) Appl. No.: 19/017,303

(22) Filed: Jan. 10, 2025

Related U.S. Application Data
(63) Continuation of application No. 16/304,501, filed on Nov. 26, 2018, now abandoned.
(60) Provisional application No. 62/342,829, filed on May 27, 2016.

(51) Int. Cl. A61K 47/64; A61K 9/00; A61K 9/51; A61K 31/337; A61K 31/704; A61K 39/00; A61K 39/395; A61P 35/00; C07K 16/32

(52) U.S. Cl. CPC A61K 47/6455; A61K 9/0019; A61K 9/51; A61K 9/5169; A61K 31/337; A61K 31/704; A61K 39/39558; A61P 35/00; C07K 16/32; A61K 2039/507; A61K 2039/55; A61K 2039/55555; C07K 2317/24

(57) ABSTRACT

Disclosed herein are compositions comprising nanoparticles comprising a carrier polypeptide and a double-stranded oligonucleotide, wherein the carrier polypeptide comprises a cell-targeting segment, a cell-penetrating segment, and an oligonucleotide-binding segment; and wherein the molar ratio of the carrier polypeptide to the double-stranded oligonucleotide in the nanoparticle composition is less than about 6:1, along with methods of making and using such nanoparticles. Further described are methods of treating a subject with a cancer, such as a chemotherapeutic drug resistant cancer comprising administering to the subject a composition comprising nanoparticles, the nanoparticles comprising a carrier polypeptide comprising a cell-targeting segment, a cell-penetrating segment, and an oligonucleotide-binding segment; a double-stranded oligonucleotide bound to the oligonucleotide-binding segment; and a chemotherapeutic drug bound to the double-stranded oligonucleotide. Also described are pharmaceutical compositions, articles of manufacture, and kits comprising the described nanoparticles.

### CROSS-REFERENCE TO RELATED APPLICATION

This application is a continuation of U.S. application Ser. No. 16/304,501, which adopts the international filing date of May 26, 2017, which is the U.S. national phase application under 35 U.S.C. 371 of International Application No. PCT/US2017/034719, filed on May 26, 2017, which claims priority benefit to U.S. Provisional Application No. 62/342,829, filed on May 27, 2016, entitled “DRUG-DELIVERY NANOPARTICLES AND TREATMENTS FOR DRUG-RESISTANT CANCER,” which is incorporated herein by reference for all purposes.

### STATEMENT REGARDING FEDERALLY SPONSORED RESEARCH

This invention was made with government support under Grant No. CA129822 awarded by the National Institute of Health. The government has certain rights in the invention.

### REFERENCE TO AN ELECTRONIC SEQUENCE LISTING

The contents of the electronic sequence listing (203782001301SEQLIST.xml; Size: 9,382 bytes; and Date of Creation: Jan. 10, 2025) is herein incorporated by reference in its entirety.

### FIELD OF THE INVENTION

The present invention relates to the methods and compositions for the treatment of cancer, including chemotherapeutic drug-resistant cancer.

### BACKGROUND

Cancer resistance to chemotherapeutic drug treatment, such as small molecule chemotherapy agents or antibody chemotherapeutic agents, can occur due to the type of cancer or can arise after drug exposure. Drug resistance can arise, for example, by alterations of drug metabolism or variations in the expression of drug targets, such as cell surface receptors. Increased dosage of the drug is only effective to a certain limit, and in many cases enhances undesired side effects of the drug. Thus, in many cases, drug therapies are only effective for a certain period of time, if at all, for a patient or a particular cancer type before the drug losses its effectiveness.

Doxorubicin is an exemplary small molecule chemotherapeutic drug that exerts its therapeutic effect by intercalating the DNA of replicating cells, and preventing their division. However, doxorubicin has several adverse events, the most prominent being of cardiac nature and hand-foot syndrome, which limit its use and/or the upper dose for administration to humans. Several attempts have been made to make doxorubicin more patient-friendly. One of the most successful formulations of doxorubicin is a liposomal formulation, commercialized as DOXIL® or generic liposomal doxorubicin as “LipoDox”. However, this formulation suffers from shortcomings that limit the use of doxorubicin in the treatment of diseases that should respond to its administration.

Trastuzumab, marketed as Herceptin®, is an antibody chemotherapeutic agent that binds to HER2, present on the surface of many (but not all) breast cancer cell types. However, trastuzumab-resistant cancers can also arise after the start of treatment, limiting the efficacy of the therapeutic.

The disclosures of all publications, patents, and patent applications referred to herein are hereby incorporated herein by reference in their entireties.

### SUMMARY OF THE INVENTION

Described here is a composition comprising nanoparticles comprising a carrier polypeptide and a double-stranded oligonucleotide, wherein the carrier polypeptide comprises a cell-targeting segment, a cell-penetrating segment, and an oligonucleotide-binding segment; and wherein the molar ratio of the carrier polypeptide to the double-stranded oligonucleotide in the nanoparticle composition is less than about 6:1. In some embodiments, the molar ratio of the carrier polypeptide to the double-stranded oligonucleotide in the nanoparticle composition is about 4:1 to less than about 6:1. In some embodiments, the molar ratio of the carrier polypeptide to the double-stranded oligonucleotide in the nanoparticle composition is about 4:1.

In some embodiments, the average size of the nanoparticles in the composition is no greater than about 50 nm.

In some embodiments, the molar ratio of the carrier polypeptide to the double-stranded oligonucleotide in the nanoparticles is less than about 6:1. In some embodiments, the molar ratio of the carrier polypeptide to the double-stranded oligonucleotide in the nanoparticles is about 4:1 to less than about 6:1. In some embodiments, the molar ratio of the carrier polypeptide to the double-stranded oligonucleotide in the nanoparticles is about 4:1 or about 5:1. In some embodiments, the molar ratio of the carrier polypeptide to the double-stranded oligonucleotide in the nanoparticles is about 4:1.

In some embodiments, the double-stranded oligonucleotide is DNA. In some embodiments, the double-stranded oligonucleotide is RNA. In some embodiments, the double-stranded oligonucleotide is about 10 base pairs to about 100 base pairs in length. In some embodiments, the double-stranded oligonucleotide is about 20 to about 50 base pairs in length.

In some embodiments, the double-stranded oligonucleotide is bound to a small-molecule drug. In some embodiments, the small-molecule drug intercalates the double-stranded oligonucleotide. In some embodiments, the molar ratio of the double-stranded oligonucleotide to the small-molecule drug in the nanoparticle composition is about 1:1 to about 1:60. In some embodiments, the small-molecule drug is a chemotherapeutic agent. In some embodiments, the small-molecule drug is an anthracycline. In some embodiments, the small-molecule drug is doxorubicin.

In some embodiments, the cell-targeting segment binds a mammalian cell. In some embodiments, the cell-targeting segment binds a diseased cell. In some embodiments, the cell-targeting segment binds a cancer cell. In some embodiments, the cell-targeting segment binds HER3 expressed on the surface of a cell. In some embodiments, the cell-targeting segment comprises a heregulin sequence or a variant thereof.

In some embodiments, the cell-penetrating segment comprises a penton base polypeptide or a variant thereof. In some embodiments, the penton base segment comprises a mutant penton base polypeptide. In some embodiments, the penton base segment comprises a truncated penton base polypeptide.

In some embodiments, the oligonucleotide-binding segment is positively charged. In some embodiments, the oligonucleotide-binding segment comprises polylysine. In some embodiments, the oligonucleotide-binding segment comprises decalysine.

In some embodiments, the composition is sterile. In some embodiments, the composition is a liquid composition. In some embodiments, the composition is a dry composition. In some embodiments, is lyophilized.

In some embodiments, there is provided an article of manufacture comprising any one of the described compositions in a vial. In some embodiments, the vial is sealed.

In some embodiments, there is provided a kit comprising any one of the described compositions and an instruction for use.

In some embodiments, there is provided a method of treating cancer in a subject comprising administering a composition described herein to the subject. In some embodiments, the cancer is a HER3+ cancer. In some embodiments, the cancer is a drug-resistant cancer. In some embodiments, the cancer is breast cancer, glial cancer, ovarian cancer, or prostate cancer. In some embodiments, the cancer is triple-negative breast cancer. In some embodiments, the cancer is metastatic. In some embodiments, the cancer is resistant to a HER2+ antibody chemotherapeutic agent, lapatinib, or an anthracycline. In some embodiments, the cancer is resistant to doxorubicin or liposomal doxorubicin. In some embodiments, the cancer is resistant to trastuzumab or pertuzumab. In some embodiments, the cancer is resistant to lapatinib.

Also provided herein there is a method of killing a chemotherapeutic drug-resistant cancer cell comprising contacting the chemotherapeutic drug-resistant cancer cell with a plurality of nanoparticles, the nanoparticles comprising a carrier polypeptide comprising a cell-targeting segment, a cell-penetrating segment, and an oligonucleotide-binding segment; a double-stranded oligonucleotide bound to the oligonucleotide-binding segment; and a chemotherapeutic drug bound to the double-stranded oligonucleotide.

In another aspect provided herein, there is a method of treating a subject with a chemotherapeutic drug-resistant cancer, comprising administering to the subject a composition comprising nanoparticles, the nanoparticles comprising a carrier polypeptide comprising a cell-targeting segment, a cell-penetrating segment, and an oligonucleotide-binding segment; a double-stranded oligonucleotide bound to the oligonucleotide-binding segment; and a chemotherapeutic drug bound to the double-stranded oligonucleotide.

In some embodiments, the chemotherapeutic drug is intercalated into the double-stranded oligonucleotide.

In some embodiments, the chemotherapeutic drug-resistant cancer is a HER3+ cancer. In some embodiments, the chemotherapeutic drug-resistant cancer is breast cancer, glial cancer, ovarian cancer, or prostate cancer. In some embodiments, the chemotherapeutic drug-resistant cancer is triple-negative breast cancer. In some embodiments, the chemotherapeutic drug-resistant cancer is metastatic. In some embodiments, the chemotherapeutic drug-resistant cancer is resistant to an anthracycline or lapatinib. In some embodiments, the chemotherapeutic drug-resistant cancer is resistant to doxorubicin or liposomal doxorubicin. In some embodiments, the chemotherapeutic drug-resistant cancer cell is resistant to a HER2+ antibody chemotherapeutic agent. In some embodiments, the chemotherapeutic drug-resistant cancer cell is resistant to trastuzumab or pertuzumab.

In some embodiments, the chemotherapeutic agent is an anthracycline. In some embodiments, the chemotherapeutic agent is doxorubicin.

In some embodiments, the average size of the nanoparticles is no greater than about 50 nm.

In some embodiments, the double stranded oligonucleotide is DNA. In some embodiments, the double stranded oligonucleotide is RNA.

In another aspect, there is provided a method of making a nanoparticle composition comprising combining a carrier polypeptide and a double-stranded oligonucleotide at a molar ratio of less than about 6:1, thereby forming a plurality of nanoparticles; wherein the carrier polypeptide comprises a cell-targeting segment, a cell-penetrating segment, and an oligonucleotide-binding segment. In some embodiments, the molar ratio of the carrier polypeptide to the double-stranded oligonucleotide is about 4:1 to less than about 6:1. In some embodiments, the molar ratio of the carrier polypeptide to the double-stranded oligonucleotide is about 4:1.

In some embodiments, the average size of the nanoparticles is no greater than about 50 nm.

In some embodiments, the method further comprises combining the double-stranded oligonucleotide and a small-molecule drug prior to combining the double-stranded oligonucleotide and the carrier polypeptide. In some embodiments, the small-molecule drug intercalates into the double-stranded oligonucleotide. In some embodiments, the double-stranded oligonucleotide and the small-molecule drug are combined at a molar ratio of about 1:1 to about 1:60. In some embodiments, the double-stranded oligonucleotide and the small-molecule drug are combined at a molar ratio of about 1:10 or about 1:40.

In some embodiments, the method further comprises separating unbound small-molecule drug from the double-stranded oligonucleotide prior to combining the double-stranded oligonucleotide and the carrier polypeptide.

In some embodiments, the method further comprises separating unbound carrier polypeptide or unbound double-stranded oligonucleotide from the plurality of nanoparticles.

In some embodiments, the method further comprises concentrating the nanoparticle composition.

In some embodiments, the double-stranded oligonucleotide is DNA. In some embodiments, the double-stranded oligonucleotide is RNA. In some embodiments, the double-stranded oligonucleotide is about 10 base pairs to about 100 base pairs in length. In some embodiments, the double-stranded oligonucleotide is about 20 to about 50 base pairs in length.

In some embodiments, the small-molecule drug is a chemotherapeutic agent. In some embodiments, the small-molecule drug is an anthracycline. In some embodiments, the small-molecule drug is doxorubicin.

In some embodiments, the cell-targeting segment comprises a heregulin sequence or a variant thereof. In some embodiments, the cell-penetrating segment comprises a penton base polypeptide or a variant thereof. In some embodiments, the penton base segment comprises a mutant penton base. In some embodiments, the penton base segment comprises a truncated penton base. In some embodiments, the oligonucleotide-binding segment is positively charged. In some embodiments, the oligonucleotide-binding segment comprises polylysine. In some embodiments, the oligonucleotide-binding segment comprises decalysine.

Further provided is a nanoparticle composition made according to any one of the methods described herein.

### BRIEF DESCRIPTION OF THE DRAWINGS

FIG. 1 illustrates a schematic of the carrier polypeptide comprising a cell-targeting domain, a cell-penetrating domain, and an oligonucleotide-binding domain. When carrier polypeptides are combined with the double stranded oligonucleotides, nanoparticles are formed. Optionally, the double-stranded oligonucleotide is pre-bound to a small molecule drug.

FIG. 2 presents average particle size (as determined by dynamic light scattering) after combining an exemplary HerPBK10 carrier polypeptide with double stranded DNA oligonucleotides (bound with doxorubicin) at a 2:1, 3:1, 4:1, 5:1, or 6:1 molar ratio. The HerPBK10 alone and doxorubicin-bound double stranded oligonucleotide alone is shown as a comparison.

FIG. 3 shows cryo-electron microscopy (“cryoEM”) images of nanoparticles formed after combining doxorubicin-bound double stranded DNA oligonucleotides with an exemplary HerPBK10 carrier polypeptide at a molar ratio of 4:1:10, 4:1:40, and 6:1:10 (HerPBK10:dsDNA:doxorubicin). The formed particles are of approximately equal size and morphology.

FIG. 4 shows the effect on MDA-MB-435 human cancer cell survival after exposure to nanoparticles with either no doxorubicin (4:1 molar ratio of HerPBK10:dsDNA, referred to as “Empty Eosomes”), nanoparticles with a 4:1:40 molar ratio of HerPBK10:dsDNA:doxorubicin (referred to as “Eos-001 (4:1:40)”), nanoparticles with a 6:1:10 molar ratio of HerPBK10:dsDNA:doxorubicin (referred to as “Eos-001 (6:1:10)”), or LipoDox. Concentration of the drug refers to concentration of doxorubicin. The input of “Empty Eosomes” was normalized based on the relative protein content in the EOS-001 (4:1:40) at various EOS-001 treatment concentrations. The inset figure presents the relative amounts of HER1, HER2, HER3, and HER4 on the surface of the MDA-MB-435 cells.

FIG. 5A shows the effect on BT474 human breast cancer cell survival after exposure to nanoparticles with either no doxorubicin (4:1 molar ratio of HerPBK10:dsDNA, referred to as “Empty Eosomes”), nanoparticles with a 4:1:40 molar ratio of HerPBK10:dsDNA:doxorubicin (referred to as “Eos-001 (4:1:40)”), nanoparticles with a 6:1:10 molar ratio of HerPBK10:dsDNA:doxorubicin (referred to as “Eos-001 (6:1:10)”), or LipoDox. Concentration of the drug refers to concentration of doxorubicin (or, in the case of the “Empty Eosomes” an equivalent amount of doxorubicin present in the Eos-001 (4:1:40) for the same amount of HerPBK10 carrier polypeptide). The inset figure presents the relative amounts of HER1, HER2, HER3, and HER4 on the surface of the BT474 cells.

FIG. 5B shows the effect on BT474-R trastuzumab-resistant human breast cancer cell survival after exposure to nanoparticles with either no doxorubicin (4:1 molar ratio of HerPBK10:dsDNA, referred to as “Empty Eosomes (4:1)”; or 6:1 molar ratio of HerPBK10:dsDNA, referred to as “Empty Eosomes (6:1)”), nanoparticles with a 4:1:40 molar ratio of HerPBK10:dsDNA:doxorubicin (referred to as “Eos-001 (4:1:40)”), nanoparticles with a 6:1:10 molar ratio of HerPBK10:dsDNA:doxorubicin (referred to as “Eos-001 (6:1:10)”), or LipoDox. Concentration of the drug refers to concentration of doxorubicin (or, in the case of the “Empty Eosomes (4:1)” an equivalent amount of doxorubicin present in the Eos-001 (4:1:40) for the same amount of HerPBK10 carrier polypeptide, and in the case of the “Empty Eosomes (6:1)” an equivalent amount of doxorubicin present in the Eos-001 (6:1:10) for the same amount of HerPBK10 carrier polypeptide). The inset figure presents the relative amounts of HER1, HER2, HER3, and HER4 on the surface of the BT474 cells and BT474-R cells.

FIG. 6 shows the effect on JIMT1 human breast cancer cell survival after exposure to nanoparticles with either no doxorubicin (4:1 molar ratio of HerPBK10:dsDNA, referred to as “Empty Eosomes (4:1)”; or 6:1 molar ratio of HerPBK10:dsDNA, referred to as “Empty Eosomes (6:1)”), nanoparticles with a 4:1:40 molar ratio of HerPBK10:dsDNA:doxorubicin (referred to as “Eos-001 (4:1:40)”), nanoparticles with a 6:1:10 molar ratio of HerPBK10:dsDNA:doxorubicin (referred to as “Eos-001 (6:1:10)”), or LipoDox. Concentration of the drug refers to concentration of doxorubicin (or, in the case of the “Empty Eosomes (4:1)” an equivalent amount of doxorubicin present in the Eos-001 (4:1:40) for the same amount of HerPBK10 carrier polypeptide, and in the case of the “Empty Eosomes (6:1)” an equivalent amount of doxorubicin present in the Eos-001 (6:1:10) for the same amount of HerPBK10 carrier polypeptide). The inset figure presents the relative amounts of HER1, HER2, HER3, and HER4 on the surface of the JIMT1 cells.

FIG. 7 shows the effect on U251 human glioma cell survival after exposure to nanoparticles with either no doxorubicin (4:1 molar ratio of HerPBK10:dsDNA, referred to as “Empty Eosomes (4:1)”; or 6:1 molar ratio of HerPBK10:dsDNA, referred to as “Empty Eosomes (6:1)”), nanoparticles with a 4:1:40 molar ratio of HerPBK10:dsDNA:doxorubicin (referred to as “Eos-001 (4:1:40)”), nanoparticles with a 6:1:10 molar ratio of HerPBK10:dsDNA:doxorubicin (referred to as “Eos-001 (6:1:10)”), or LipoDox. Concentration of the drug refers to concentration of doxorubicin (or, in the case of the “Empty Eosomes (4:1)” an equivalent amount of doxorubicin present in the Eos-001 (4:1:40) for the same amount of HerPBK10 carrier polypeptide, and in the case of the “Empty Eosomes (6:1)” an equivalent amount of doxorubicin present in the Eos-001 (6:1:10) for the same amount of HerPBK10 carrier polypeptide). The inset figure presents the relative amounts of HER1, HER2, HER3, and HER4 on the surface of the U251 cells.

FIG. 8 shows the effect on A2780-ADR doxorubicin-resistant human ovarian cancer cell survival after exposure to nanoparticles with either no doxorubicin (4:1 molar ratio of HerPBK10:dsDNA, referred to as “Empty Eosomes”), nanoparticles with a 4:1:40 molar ratio of HerPBK10:dsDNA:doxorubicin (referred to as “Eos-001 (4:1:40)”), or LipoDox. Concentration of the drug refers to concentration of doxorubicin (or, in the case of the “Empty Eosomes” an equivalent amount of doxorubicin present in the Eos-001 (4:1:40) for the same amount of HerPBK10 carrier polypeptide).

FIG. 9 shows the effect on 4T1 mouse triple-negative mammary cancer cell survival after exposure to nanoparticles with either no doxorubicin (4:1 molar ratio of HerPBK10:dsDNA, referred to as “Empty Eosomes”), nanoparticles with a 4:1:40 molar ratio of HerPBK10:dsDNA:doxorubicin (referred to as “Eos-001 (4:1:40)”), or LipoDox. Concentration of the drug refers to concentration of doxorubicin (or, in the case of the “Empty Eosomes” an equivalent amount of doxorubicin present in the Eos-001 (4:1:40) for the same amount of HerPBK10 carrier polypeptide).

FIG. 10 shows the effect on SKOV3 human ovarian cancer cell survival after exposure to nanoparticles with either no doxorubicin (4:1 molar ratio of HerPBK10:dsDNA, referred to as “Empty Eosomes”), nanoparticles with a 4:1:40 molar ratio of HerPBK10:dsDNA:doxorubicin (referred to as “Eos-001 (4:1:40)”), or LipoDox. Concentration of the drug refers to concentration of doxorubicin (or, in the case of the “Empty Eosomes” an equivalent amount of doxorubicin present in the Eos-001 (4:1:40) for the same amount of HerPBK10 carrier polypeptide).

FIG. 11A shows the effect on LNCaP-GFP human prostate cancer cell survival after exposure to nanoparticles with either no doxorubicin (4:1 molar ratio of HerPBK10:dsDNA, referred to as “Empty Eosomes”), nanoparticles with a 4:1:40 molar ratio of HerPBK10:dsDNA:doxorubicin (referred to as “Eos-001 (4:1:40)”), or LipoDox. Concentration of the drug refers to concentration of doxorubicin (or, in the case of the “Empty Eosomes” an equivalent amount of doxorubicin present in the Eos-001 (4:1:40) for the same amount of HerPBK10 carrier polypeptide).

FIG. 11B shows the effect on RANKL human bone-metastatic prostate cancer cell survival after exposure to nanoparticles with either no doxorubicin (4:1 molar ratio of HerPBK10:dsDNA, referred to as “Empty Eosomes”), nanoparticles with a 4:1:40 molar ratio of HerPBK10:dsDNA:doxorubicin (referred to as “Eos-001 (4:1:40)”), or LipoDox. Concentration of the drug refers to concentration of doxorubicin (or, in the case of the “Empty Eosomes” an equivalent amount of doxorubicin present in the Eos-001 (4:1:40) for the same amount of HerPBK10 carrier polypeptide).

FIG. 11C shows the relative amounts of HER1, HER2, HER3, and HER4 expressed on the surface of LNCaP-GFP human prostate cancer cells and RANKL human bone-metastatic prostate cancer cells

FIG. 12A shows the effect on BT549 human triple-negative breast cancer cell survival after exposure to nanoparticles with either no doxorubicin (4:1 molar ratio of HerPBK10:dsDNA, referred to as “Empty Eosomes”), nanoparticles of Eos-001 (4:1:40 HerPBK10:dsDNA:doxorubicin), or LipoDox. Concentration of the drug refers to concentration of doxorubicin (or, in the case of the “Empty Eosomes” the input was normalized based on the relative protein content in the Eos-001 (4:1:40) at various Eos-001 treatment.

FIG. 12B shows the relative expression of HER1, HER 2, HER3, and HER4 in BT549 cells.

FIG. 13 shows the effect of Eos-001 nanoparticles (HerPBK10, dsDNA, and doxorubicin), trastuzumab, or the combination of trastuzumab and pertuzumab on BT474 or BT474-TR cells.

FIG. 14 shows the relative cell survival of trastuzumab resistant BT474-TR cells after treatment with pertuzumab, trastuzumab, Eos-001 nanoparticles (HerPBK10, dsDNA, and doxorubicin), a combination of Eos-001 nanoparticles and pertuzumab, or Eos-001 nanoparticles after a 4 hour pre-treatment with pertuzumab.

FIG. 15A shows relative cell surface levels of HER3 in parental (i.e., non-trastuzumab resistant) cells and trastuzumab resistant cells for BT474 and SKBR 3 cell lines. HER3 is overexpressed in the trastuzumab resistant cell lines relative to the parental cell lines.

FIG. 15B shows the contribution of HER3 to targeted toxicity of Eos-001 nanoparticles (HerPBK10, dsDNA, and doxorubicin). Trastuzumab-resistant or non-trastuzumab resistant BT474 or SKBR3 cells were treated with Eos-001 nanoparticles with or without a human HER3 blocking peptide (Prospec).

FIG. 16 illustrates relative cell survival of non-trastuzumab resistant cell lines (SKBR3 (FIG. 16A), BT474 (FIG. 16B), or MDA-MB-435 (FIG. 16C)) and trastuzumab-resistant cell lines (SKBR3-TR (FIG. 16D) and BT474-TR (FIGS. 16E and 16F)) in response to treatment with trastuzumab, Eos-001 nanoparticles (HerPBK10, dsDNA, and doxorubicin), or Eos-001 nanoparticles after 4 or 24 hours of pre-treatment with trastuzumab.

FIG. 17 shows relative cell survival of BT-474 or SKBR3 cells, or trastuzumab-resistant BT474-TR, SKBR3-TR, or JIMT-1 cells in response to treatment with lapatinib or Eos-001 nanoparticles (HerPBK10, dsDNA, and doxorubicin).

### DETAILED DESCRIPTION OF THE EMBODIMENTS

Described herein are nanoparticle compositions comprising nanoparticles comprising a carrier polypeptide and a double-stranded oligonucleotide, wherein the carrier polypeptide comprises a cell-targeting segment, a cell-penetrating segment, and an oligonucleotide-binding segment.

In one aspect, there is provided a nanoparticle composition comprising nanoparticles comprising a carrier polypeptide and a double-stranded oligonucleotide, the carrier polypeptide comprises a cell-targeting segment, a cell-penetrating segment, and an oligonucleotide-binding segment; wherein the molar ratio of the carrier polypeptide to the double-stranded oligonucleotide in the nanoparticle composition is less than about 6:1. Optionally, a small molecule drug, such as a chemotherapeutic drug, is bound to the double-stranded oligonucleotide. Combining the carrier polypeptide and the double stranded oligonucleotide results in the formation of stable nanoparticles. As further described herein, it has been found that these stable nanoparticles can be formed even when the molar ratios of carrier polypeptide to double stranded oligonucleotide in the composition (and/or in the nanoparticles) is less than 6:1. The nanoparticle composition can be useful, for example, in the treatment of cancer, including chemotherapeutic drug resistant cancers.

In another aspect, there is provided a method of treating a subject with a chemotherapeutic drug-resistant cancer, comprising administering to the subject a composition comprising nanoparticles, the nanoparticles comprising a carrier polypeptide comprising a cell-targeting segment, a cell-penetrating segment, and an oligonucleotide-binding segment; a double-stranded oligonucleotide bound to the oligonucleotide-binding segment; and a chemotherapeutic drug bound to the double-stranded oligonucleotide. Many cancer types are resistant to certain chemotherapeutic drugs, such as doxorubicin, lapatinib, or HER2+ antibodies (such as trastuzumab or pertuzumab). Increased concentration of the drug often fails to be effective and can result in significant undesirable side effects. As further described herein, the nanoparticle compositions can be used to kill chemotherapeutic drug resistant cancer cells and treat patients with chemotherapeutic drug resistant cancers.

Doxorubicin is an exemplary chemotherapeutic drug that can be used to treat various malignancies. However, its utility is limited by the drug efflux mechanisms in the cell. Higher doses of doxorubicin to overcome the cellular efflux challenges are generally unadvisable due to significant side effects, including cardiomyopathy. Liposomal doxorubicin (also referred to as “LipoDox”) has also been used to enhance cellular uptake, but significant side effects after administration continue.

It has been found that compositions comprising the nanoparticles described herein are more effective at killing targeted cancer cells than liposomal doxorubicin. The nanoparticles are also effective at killing cancer cells that are resistant to chemotherapeutic drugs, including antibodies (such as an anti-HER2 antibody, namely trastuzumab) or small molecule chemotherapeutic agents, such as doxorubicin (for example LipoDox). Thus, the nanoparticles and compositions described herein are particularly useful for the treatment of cancer, including chemotherapeutic drug resistant cancers.

### Definitions

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X”.

The term “effective” is used herein, unless otherwise indicated, to describe an amount of a compound or component which, when used within the context of its use, produces or effects an intended result, whether that result relates to the treatment of an infection or disease state or as otherwise described herein.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available pairwise sequence computer software. Those skilled in the art can determine appropriate parameters for aligning sequences, including any algorithms needed to achieve maximal alignment over the full length of the sequences being compared. The % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

The term “pharmaceutically acceptable” as used herein means that the compound or composition is suitable for administration to a subject, including a human subject, to achieve the treatments described herein, without unduly deleterious side effects in light of the severity of the disease and necessity of the treatment.

The term “subject” or “patient” is used synonymously herein to describe a mammal. Examples of a subject include a human or animal (including, but not limited to, dog, cat, rodent (such as mouse, rat, or hamster), horse, sheep, cow, pig, goat, donkey, rabbit, or primates (such as monkey, chimpanzee, orangutan, baboon, or macaque)).

The terms “treat,” “treating,” and “treatment” are used synonymously herein to refer to any action providing a benefit to a subject afflicted with a disease state or condition, including improvement in the condition through lessening, inhibition, suppression, or elimination of at least one symptom, delay in progression of the disease, delay in recurrence of the disease, or inhibition of the disease.

It is understood that aspects and variations of the invention described herein include “consisting” and/or “consisting essentially of” aspects and variations.

It is to be understood that one, some or all of the properties of the various embodiments described herein may be combined to form other embodiments of the present invention.

The section headings used herein are for organizational purposes only and are not to be construed as limiting the subject matter described.

### Nanoparticle Compositions

The nanoparticle compositions described herein comprise a carrier polypeptide, which comprises a cell-targeting segment, a cell-penetrating segment, and an oligonucleotide-binding segment. The nanoparticles further comprise a double-stranded oligonucleotide. The double stranded oligonucleotide can bind the oligonucleotide-binding segment. In some embodiments, a small molecule drug is bound to the double stranded oligonucleotide. In some embodiments, the ratio of carrier polypeptide to double stranded oligonucleotide in the composition is less than about 6:1.

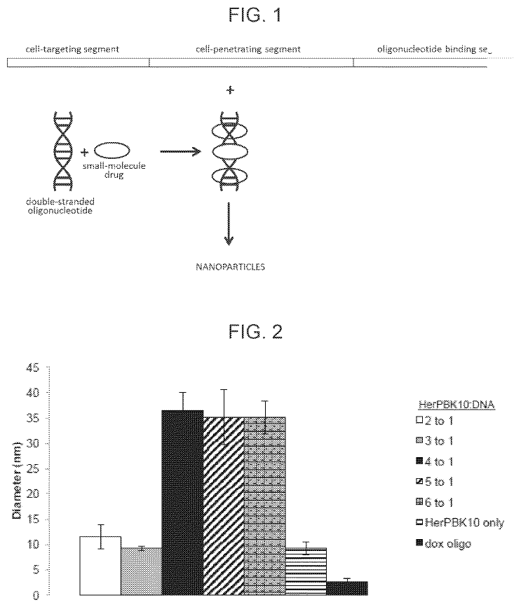

The cell-targeting segment, the cell-penetrating segment, and the oligonucleotide-binding segment are fused together in a single carrier polypeptide. The segments described herein are modular, and can be combined in various combinations. That is, a carrier polypeptide can comprise any of the described cell-targeting segments, the cell-penetrating segments, or the oligonucleotide-binding segments. FIG. 1 illustrates a carrier peptide with a cell-targeting segment, a cell-penetrating segment, and an oligonucleotide-binding segment. As further shown in FIG. 1, combining the carrier peptide with the double stranded oligonucleotide results in the formation of nanoparticles. Optionally, the double-stranded oligonucleotide is pre-bound to a small-molecule drug prior to forming the nanoparticles.

The nanoparticles can be formed by combining the carrier polypeptide with a double-stranded oligonucleotide. In some embodiments, the carrier polypeptide is combined with the double-stranded oligonucleotide at a molar ratio of less than about 6:1 (for example, about 4:1 to less than about 6:1, such as about 4:1 to about 4.5:1, about 4.5:1 to about 5:1, about 5:1 to about 5.5:1, about 5.5:1 to less than about 6:1, about 4:1, about 4.5:1, about 5:1, or about 5.5), thereby forming a nanoparticle composition. Thus, in some embodiments, the nanoparticle composition comprises carrier polypeptides and double stranded oligonucleotides at a molar ratio of less than about 6:1 (such as about 4:1 to less than about 6:1, such as about 4:1 to about 4.5:1, about 4.5:1 to about 5:1, about 5:1 to about 5.5:1, about 5.5:1 to less than about 6:1, about 4:1, about 4.5:1, about 5:1, or about 5.5:1). A ratio of components in the nanoparticle composition refers to the total ratio of components in the composition, without regard to whether those components assemble into nanoparticles.

In some embodiments, the nanoparticle composition comprises nanoparticles with a homogenous molar ratio of carrier polypeptides to double-stranded oligonucleotides. In some embodiments, the nanoparticles comprise carrier polypeptides and double-stranded oligonucleotides at a molar ratio of about 6:1, about 5:1, or about 4:1. The ratio of components in the nanoparticles can be determined by separating the nanoparticles from the balance of the composition (for example, by centrifuging the composition and decanting the supernatant), and measuring the components in the isolated nanoparticles.

The cell-targeting segment can bind to a molecule present on the surface of a cell. Binding of the molecule by the cell-targeting segment allows the nanoparticle to be targeted to the cell. Thus, the targeted molecule present on the cell can depend on the targeted cell. In some embodiments, the targeted molecule is an antigen, such as a cancer antigen. In some embodiments, the cell-targeting segment is an antibody, an antibody fragment, a cytokine, or a receptor ligand.

In some embodiments, the cell-targeting segment binds to a target on the surface of a targeted cell. For example, in some embodiments, the cell-targeting segment binds to a cell surface protein, such as a receptor. In some embodiments, the cell-targeting segment binds to of 4-IBB, 5T4, adenocarcinoma antigen, alpha-fetoprotein, BAFF, C242 antigen, CA-125, carbonic anhydrase 9 (CA-IX), c-MET, CCR4, CD152, CD19, CD20, CD200, CD22, CD221, CD23 (IgE receptor), CD28, CD30 (TNFRSF8), CD33, CD4, CD40, CD44v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, hepatocyte growth factor (HGF), human scatter factor receptor kinase, IGF-1 receptor, IGF-I, IgG1, LI-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, vimentin, Internalin B, bacterial invasin (Inv) protein, or a fragment thereof.

In some embodiment, the cell-targeting segment is heregulin or a receptor binding fragment thereof, and can be referred to as “Her”. The heregulin can be, for example, heregulin-α. SEQ ID NO: 2 is an exemplary wild-type Her sequence. In some embodiments, the cell-targeting segment is SEQ ID NO: 2, or a polypeptide that has about 80% or greater, about 85% or greater, about 90% or greater, about 92% or greater, about 93% or greater, about 94% or greater, about 95% or greater, about 96% or greater, about 97% or greater, about 98% or greater, or about 99% or greater amino acid sequence identity to SEQ ID NO: 2. In some embodiments, the cell-targeting segment binds a heregulin receptor, for example HER3. In some embodiments, the cell-targeting segment is a truncation of SEQ ID NO: 2, such as having about 50% or less, about 60% or less, about 70% or less, about 80% or less, about 90% or less, or about 95% or less of the length of SEQ ID NO:2. In some embodiments, the cell-targeting segment has a length of between about 50% and about 100% of SEQ ID NO: 1 (such as between about 60% and about 95%, or between about 70% and 90% of SEQ ID NO: 1). The cell-targeting segment truncation retains the HER3 targeting properties.

In some embodiments, the cell targeted by the cell-targeting segment is a mammalian cell, such as a human cell. In some embodiments, the cell is a diseased cell, such as a cancer cell. In some embodiment, the cell is a HER3+ cancer cell. In some embodiment, the cell is a breast cancer cell (for example, a triple negative breast cancer cell), a glial cancer cell, an ovarian cancer cell, or a prostate cancer cell. The cell-targeting segment can bind a molecule present on the surface of the targeted cell, which targets the nanoparticle to the targeted cell.

The cell-penetrating segment of the carrier polypeptide facilitates entry of the nanoparticle into the cell targeted by the cell-targeting segment. In some embodiments, the cell-penetrating segment comprises (and, in some embodiments, is) a penton base (“PB”) protein, or a variant thereof. By way of example, in some embodiments, the cell-penetrating segment comprises (and, in some embodiments, is) the adenovirus serotype 5 (Ad5) penton base protein. In some embodiments, the cell-penetrating segment comprises (and, in some embodiments, is) a penton base protein with an amino acid variation or deletion. The amino acid variation can be a conservative mutation. In some embodiments, the cell-penetrating segment is a truncated penton base protein. SEQ ID NO: 1 is an exemplary penton base protein. In some embodiments, the cell-penetrating segment in SEQ ID NO: 1 or a polypeptide that has about 80% or greater, about 85% or greater, about 90% or greater, about 92% or greater, about 93% or greater, about 94% or greater, about 95% or greater, about 96% or greater, about 97% or greater, about 98% or greater, or about 99% or greater amino acid sequence identity to SEQ ID NO: 1. In some embodiments, the cell-penetrating segment is a truncation of SEQ ID NO: 1, such as having about 50% or less, about 60% or less, about 70% or less, about 80% or less, about 90% or less, or about 95% or less of the length of SEQ ID NO: 1. In some embodiments, the cell-penetrating segment has a length of between about 50% and about 100% of SEQ ID NO: 1 (such as between about 60% and about 95%, or between about 70% and 90% of SEQ ID NO: 1).

The cell-penetrating segment can comprise one or more variants that enhance subcellular localization of the carrier polypeptide. For example, in some embodiments, the cell-penetrating segment comprises one or more variants which cause the carrier polypeptide to preferentially localize in the cytoplasm or the nucleus. In embodiments, where the carrier polypeptide is bound to an oligonucleotide (which may itself be bound to a small molecule drug), the variant cell-penetrating segment preferentially localizes the oligonucleotide and/or small molecule drug to the cytoplasm or the nucleus. Preferential subcellular localization can be particular beneficial for certain small molecule drugs. For example, many chemotherapeutic agents function by binding to DNA localized in the cancer cell nucleus. By preferentially targeting the nucleus, the associated drug is concentrated at the location it functions. Other small molecule drugs may function in the cytoplasm, and preferentially targeting to the cytoplasm can enhance drug potency.

Exemplary cell-penetrating segment mutations that enhance subcellular localization are discussed in WO 2014/022811. The Leu60Trp mutation in the penton base protein has been shown to preferentially localize to the cytoplasm of the cell. Thus, in some embodiments, the cell-penetrating segment is a penton base protein comprising the Leu60Trp mutation. The Lys375Glu, Val449Met, and Pro469Ser mutations have been shown to preferentially localize to the nucleus of the cell. Thus, in some embodiments, the cell-penetrating segment is a penton base protein comprising a Lys375Glu, Val449Met, or Pro469Ser mutations. In some embodiments, the cell-penetrating segment is a penton base protein comprising the Lys375Glu, Val449Met, and Pro469Ser mutations. Amino acid numbering is made in reference to the wild-type penton base polypeptide of SEQ ID NO: 1.

The oligonucleotide-binding segment binds the double-stranded oligonucleotide component of the nanoparticle. The oligonucleotide-binding segment can bind the double-stranded oligonucleotide, for example, through electrostatic bonds, hydrogen bonds, or ionic bonds. In some embodiments, the oligonucleotide-binding segment is a DNA binding domain or a double-stranded RNA binding domain. In some embodiments, the oligonucleotide-binding segment is a cationic domain. In some embodiments, the oligonucleotide binding domain comprises is a polylysine sequence. In some embodiments, the oligonucleotide-binding segment is between about 3 and about 30 amino acids in length, such as between about 3 and about 10, between about 5 and about 15, between about 10 and about 20, between about 15 and about 25, or between about 20 and about 30 amino acids in length. In one exemplary embodiment, the oligonucleotide-binding segment comprises (and, in some embodiments, is) a decalysine (that is, ten sequential lysine amino acids, or “K10,” as shown in SEQ ID: 4).

Exemplary carrier polypeptides comprises Her, a penton base (or a variants thereof), and a positively charged oligonucleotide-binding segment. In some embodiments, the carrier polypeptide comprises Her, a penton base segment, and a polylysine oligonucleotide-binding segment. In some embodiment, the carrier polypeptide comprises Her, a penton base segment, and a decalysine oligonucleotide-binding segment, for example HerPBK10 (SEQ ID: 3). In some embodiments, the carrier polypeptide is a polypeptide that has about 80% or greater, about 85% or greater, about 90% or greater, about 92% or greater, about 93% or greater, about 94% or greater, about 95% or greater, about 96% or greater, about 97% or greater, about 98% or greater, or about 99% or greater amino acid sequence identity to SEQ ID NO: 3.

The carrier polypeptide associates with a double-stranded oligonucleotide to form the nanoparticle. The double-stranded oligonucleotide can be RNA or DNA. In some embodiments, the double-stranded oligonucleotide comprises a siRNA, shRNA, or microRNA. A double stranded oligonucleotide can comprise, for example, a stem-loop structure or may comprise two separate RNA strands. The double-stranded oligonucleotide need not be perfectly base paired, and in some embodiments comprises one or more bulges, loops, mismatches, or other secondary structure. In some embodiments, about 80% or more of the bases are paired, about 85% or more of the bases are paired, about 90% or more of the bases are paired, about 95% of the bases are paired, or about 100% of the bases are paired. In some embodiments, the RNA comprises a triphosphate 5′-end, such as T7-transcribed RNA. In some embodiments, the RNA is synthetically produced.

In some embodiments, the oligonucleotides are about 10 bases long to about 1000 bases long, such as about 10 bases long to about 30 bases long, about 20 bases long to about 40 bases long, about 30 bases long to about 50 bases long, about 40 bases long to about 60 bases long, about 50 bases long to about 70 bases long, about 60 bases long to about 80 bases long, about 70 bases long to about 90 bases long, about 80 bases long to about 100 bases long, about 100 bases long to about 200 bases long, about 200 bases long to about 300 bases long, about 300 bases long to about 400 bases long, about 400 bases long to about 500 bases long, about 500 bases long to about 700 bases long, or about 700 bases long to about 1000 bases long. In some embodiments, the oligonucleotides are about 25 bases long to about 35 bases long, such as about 25 bases long, about 26 bases long, about 27 bases long, about 28 bases long, about 29 bases long, about 30 bases long, about 31 bases long, about 32 bases long, about 33 bases long, about 34 bases long, or about 35 bases long.

In some embodiments, a small molecule compound (such as a small molecule drug) is bound to the double-stranded oligonucleotide, for example by electrostatic interactions or by intercalating in the double-stranded oligonucleotide. The small molecule drug can be a chemotherapeutic agent, such as doxorubicin. Other small molecule chemotherapeutic agents can include other anthracyclines (such as daunorubicin, epirubicin, idarubicin, mitoxantrone, valrubicin) alkylating or alkylating-like agents (such as carboplatin, carmustine, cisplatin, cyclophosphamide, melphalan, procarbazine, or thiotepa), or taxanes (such as paclitaxel, docetaxel, or taxotere). In some embodiments, the small molecule compound is about 1000 Daltons or less, about 900 Daltons or less, about 800 Daltons or less, about 700 Daltons or less, about 600 Daltons or less, about 500 Daltons or less, about 400 Daltons or less, or about 300 Daltons or less.

In some embodiments, the molar ratio of the small molecule drug to the double-stranded oligonucleotide in the nanoparticle composition is about 60:1 or less, such as about 50:1 or less, about 40:1 or less, about 30:1 or less, about 20:1 or less, about 10:1 or less, about 5:1 or less, about 4:1 or less, about 3:1 or less, about 2:1 or less, or about 1:1 or less. In some embodiments, the molar ratio of the small molecule drug to the double-stranded oligonucleotide in the nanoparticle composition is between about 1:1 and about 60:1, such as between about 1:1 and about 10:1, between about 5:1 and about 20:1, between about 10:1 and about 30:1, between about 20:1 and about 40:1, between about 30:1 and about 50:1, or between about 40:1 and about 60:1, about 1:1, about 1:10, about 1:20, about 1:30, about 1:40, about 1:50, or about 1:60.

In some embodiments, the nanoparticles are generally about 50 nm or less in diameter (such as about 45 nm or less, about 40 nm or less, about 35 nm or less, about 30 nm or less, about 25 nm to about 50 nm, about 25 nm to about 30 nm, about 30 nm to about 35 nm, about 35 nm to about 40 nm, or about 45 nm to about 50 nm in diameter), as measured by dynamic light scattering. The small-molecule drug, if present, is bound to the double-stranded oligonucleotide, which itself bound to the oligonucleotide-binding segment.

In some embodiments, there is provided a composition comprising nanoparticles comprising a carrier polypeptide and a double-stranded oligonucleotide (such as DNA), the carrier polypeptide comprises a cell-targeting segment, a cell-penetrating segment, and an oligonucleotide-binding segment; wherein the molar ratio of the carrier polypeptide to the double-stranded oligonucleotide in the nanoparticle composition is less than about 6:1 (such as 4:1 to less than about 6:1, or about 4:1). In some embodiments, the cell-targeting segment binds a cancer cell, such as a HER3+ cancer cell. In some embodiments, the cancer cell is a chemotherapeutic drug resistant cancer cell. In some embodiments, the double-stranded oligonucleotide is between about 20 and about 50 bases in length. In some embodiments, the double-stranded oligonucleotide is bound to a small molecule drug, such as an anthracycline (for example, doxorubicin). In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a composition comprising nanoparticles comprising a carrier polypeptide and a double-stranded oligonucleotide (such as DNA), the carrier polypeptide comprises a cell-targeting segment, a cell-penetrating segment, and an oligonucleotide-binding segment; wherein the molar ratio of the carrier polypeptide to the double-stranded oligonucleotide in the nanoparticles is less than about 6:1 (such as about 4:1 to less than about 6:1, about 5:1, or about 4:1). In some embodiments, the cell-targeting segment binds a cancer cell, such as a HER3+ cancer cell. In some embodiments, the cancer cell is a chemotherapeutic drug resistant cancer cell. In some embodiments, the double-stranded oligonucleotide is between about 20 and about 50 bases in length. In some embodiments, the double-stranded oligonucleotide is bound to a small molecule drug, such as an anthracycline (for example, doxorubicin). In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a composition comprising nanoparticles comprising a carrier polypeptide and a double-stranded oligonucleotide (such as DNA), the carrier polypeptide comprises a cell-targeting segment, a cell-penetrating segment, and an oligonucleotide-binding segment; wherein the molar ratio of the carrier polypeptide to the double-stranded oligonucleotide in the nanoparticle composition is less than about 6:1 (such as 4:1 to less than about 6:1, or about 4:1); and wherein the cell-penetrating segment comprises (and, in some embodiments, is) a penton base polypeptide or a variant thereof. In some embodiments, the cell-targeting segment binds a cancer cell, such as a HER3+ cancer cell. In some embodiments, the cancer cell is a chemotherapeutic drug resistant cancer cell. In some embodiments, the double-stranded oligonucleotide is between about 20 and about 50 bases in length. In some embodiments, the double-stranded oligonucleotide is bound to (e.g., intercalated by) a small molecule drug, such as an anthracycline (for example, doxorubicin). In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a composition comprising nanoparticles comprising a carrier polypeptide and a double-stranded oligonucleotide (such as DNA), the carrier polypeptide comprises a cell-targeting segment, a cell-penetrating segment, and an oligonucleotide-binding segment; wherein the molar ratio of the carrier polypeptide to the double-stranded oligonucleotide in the nanoparticles is less than about 6:1 (such as about 4:1 to less than about 6:1, about 5:1, or about 4:1); and wherein the cell-penetrating segment comprises (and, in some embodiments, is) a penton base polypeptide or a variant thereof. In some embodiments, the cell-targeting segment binds a cancer cell, such as a HER3+ cancer cell. In some embodiments, the cancer cell is a chemotherapeutic drug resistant cancer cell. In some embodiments, the double-stranded oligonucleotide is between about 20 and about 50 bases in length. In some embodiments, the double-stranded oligonucleotide is bound to (e.g., intercalated by) a small molecule drug, such as an anthracycline (for example, doxorubicin). In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a composition comprising nanoparticles comprising a carrier polypeptide and a double-stranded oligonucleotide (such as DNA), the carrier polypeptide comprises a cell-targeting segment, a cell-penetrating segment, and an oligonucleotide-binding segment; wherein the molar ratio of the carrier polypeptide to the double-stranded oligonucleotide in the nanoparticle composition is less than about 6:1 (such as 4:1 to less than about 6:1, or about 4:1); wherein the cell-penetrating segment comprises (and, in some embodiments, is) a penton base polypeptide or a variant thereof; and wherein the oligonucleotide-binding segment is positively charged. In some embodiments, the cell-targeting segment binds a cancer cell, such as a HER3+ cancer cell. In some embodiments, the cancer cell is a chemotherapeutic drug resistant cancer cell. In some embodiments, the double-stranded oligonucleotide is between about 20 and about 50 bases in length. In some embodiments, the double-stranded oligonucleotide is bound to (e.g., intercalated by) a small molecule drug, such as an anthracycline (for example, doxorubicin). In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a composition comprising nanoparticles comprising a carrier polypeptide and a double-stranded oligonucleotide (such as DNA), the carrier polypeptide comprises a cell-targeting segment, a cell-penetrating segment, and an oligonucleotide-binding segment; wherein the molar ratio of the carrier polypeptide to the double-stranded oligonucleotide in the nanoparticles is less than about 6:1 (such as about 4:1 to less than about 6:1, about 5:1, or about 4:1); wherein the cell-penetrating segment comprises (and, in some embodiments, is) a penton base polypeptide or a variant thereof; and wherein the oligonucleotide-binding segment is positively charged. In some embodiments, the cell-targeting segment binds a cancer cell, such as a HER3+ cancer cell. In some embodiments, the cancer cell is a chemotherapeutic drug resistant cancer cell. In some embodiments, the double-stranded oligonucleotide is between about 20 and about 50 bases in length. In some embodiments, the double-stranded oligonucleotide is bound to (e.g., intercalated by) a small molecule drug, such as an anthracycline (for example, doxorubicin). In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a composition comprising nanoparticles comprising a carrier polypeptide and a double-stranded oligonucleotide (such as DNA), the carrier polypeptide comprises a cell-targeting segment, a cell-penetrating segment, and an oligonucleotide-binding segment; wherein the molar ratio of the carrier polypeptide to the double-stranded oligonucleotide in the nanoparticle composition is less than about 6:1 (such as 4:1 to less than about 6:1, or about 4:1); wherein the cell-penetrating segment comprises (and, in some embodiments, is) a penton base polypeptide or a variant thereof; wherein the oligonucleotide-binding segment is positively charged; and wherein the cell-targeting segment comprises (and, in some embodiments, is) heregulin or a variant thereof. In some embodiments, the cell-targeting segment binds a cancer cell, such as a HER3+ cancer cell. In some embodiments, the cancer cell is a chemotherapeutic drug resistant cancer cell. In some embodiments, the double-stranded oligonucleotide is between about 20 and about 50 bases in length. In some embodiments, the double-stranded oligonucleotide is bound to (e.g., intercalated by) a small molecule drug, such as an anthracycline (for example, doxorubicin). In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a composition comprising nanoparticles comprising a carrier polypeptide and a double-stranded oligonucleotide (such as DNA), the carrier polypeptide comprises a cell-targeting segment, a cell-penetrating segment, and an oligonucleotide-binding segment; wherein the molar ratio of the carrier polypeptide to the double-stranded oligonucleotide in the nanoparticles is less than about 6:1 (such as about 4:1 to less than about 6:1, about 5:1, or about 4:1); wherein the cell-penetrating segment comprises (and, in some embodiments, is) a penton base polypeptide or a variant thereof; wherein the oligonucleotide-binding segment is positively charged; and wherein the cell-targeting segment comprises (and, in some embodiments, is) heregulin or a variant thereof. In some embodiments, the cell-targeting segment binds a cancer cell, such as a HER3+ cancer cell. In some embodiments, the cancer cell is a chemotherapeutic drug resistant cancer cell. In some embodiments, the double-stranded oligonucleotide is between about 20 and about 50 bases in length. In some embodiments, the double-stranded oligonucleotide is bound to (e.g., intercalated by) a small molecule drug, such as an anthracycline (for example, doxorubicin). In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a composition comprising nanoparticles comprising a carrier polypeptide and a double-stranded oligonucleotide (such as DNA), the carrier polypeptide comprises a cell-targeting segment, a cell-penetrating segment, and an oligonucleotide-binding segment; wherein the molar ratio of the carrier polypeptide to the double-stranded oligonucleotide in the nanoparticle composition is less than about 6:1 (such as 4:1 to less than about 6:1, or about 4:1); wherein the cell-penetrating segment comprises (and, in some embodiments, is) a penton base polypeptide or a variant thereof; wherein the oligonucleotide-binding segment comprises (and, in some embodiments, is) decalysine; and wherein the cell-targeting segment comprises (and, in some embodiments, is) heregulin or a variant thereof. In some embodiments, the cell-targeting segment binds a cancer cell, such as a HER3+ cancer cell. In some embodiments, the cancer cell is a chemotherapeutic drug resistant cancer cell. In some embodiments, the double-stranded oligonucleotide is between about 20 and about 50 bases in length. In some embodiments, the double-stranded oligonucleotide is bound (e.g., intercalated by) to a small molecule drug, such as an anthracycline (for example, doxorubicin). In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a composition comprising nanoparticles comprising a carrier polypeptide and a double-stranded oligonucleotide (such as DNA), the carrier polypeptide comprises a cell-targeting segment, a cell-penetrating segment, and an oligonucleotide-binding segment; wherein the molar ratio of the carrier polypeptide to the double-stranded oligonucleotide in the nanoparticles is less than about 6:1 (such as about 4:1 to less than about 6:1, about 5:1, or about 4:1); wherein the cell-penetrating segment comprises (and, in some embodiments, is) a penton base polypeptide or a variant thereof; wherein the oligonucleotide-binding segment comprises (and, in some embodiments, is) decalysine; and wherein the cell-targeting segment comprises (and, in some embodiments, is) heregulin or a variant thereof. In some embodiments, the cell-targeting segment binds a cancer cell, such as a HER3+ cancer cell. In some embodiments, the cancer cell is a chemotherapeutic drug resistant cancer cell. In some embodiments, the double-stranded oligonucleotide is between about 20 and about 50 bases in length. In some embodiments, the double-stranded oligonucleotide is bound to (e.g., intercalated by) a small molecule drug, such as an anthracycline (for example, doxorubicin). In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a composition comprising nanoparticles comprising a carrier polypeptide and a double-stranded oligonucleotide (such as DNA), the carrier polypeptide comprises a cell-targeting segment, a cell-penetrating segment, and an oligonucleotide-binding segment; wherein the molar ratio of the carrier polypeptide to the double-stranded oligonucleotide in the nanoparticle composition is less than about 6:1 (such as 4:1 to less than about 6:1, or about 4:1); wherein the cell-penetrating segment comprises (and, in some embodiments, is) a penton base polypeptide or a variant thereof; wherein the oligonucleotide-binding segment comprises (and, in some embodiments, is) decalysine; and wherein the cell-targeting segment comprises (and, in some embodiments, is) heregulin or a variant thereof; and wherein a chemotherapeutic drug (such as doxorubicin) is intercalated into the double-stranded oligonucleotide. In some embodiments, the cell-targeting segment binds a cancer cell, such as a HER3+ cancer cell. In some embodiments, the cancer cell is a chemotherapeutic drug resistant cancer cell. In some embodiments, the double-stranded oligonucleotide is between about 20 and about 50 bases in length. In some embodiments, the molar ratio of the chemotherapeutic drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a composition comprising nanoparticles comprising a carrier polypeptide and a double-stranded oligonucleotide (such as DNA), the carrier polypeptide comprises a cell-targeting segment, a cell-penetrating segment, and an oligonucleotide-binding segment; wherein the molar ratio of the carrier polypeptide to the double-stranded oligonucleotide in the nanoparticles is less than about 6:1 (such as about 4:1 to less than about 6:1, about 5:1, or about 4:1); wherein the cell-penetrating segment is a penton base polypeptide or a variant thereof; wherein the oligonucleotide-binding segment is decalysine; wherein the cell-targeting segment is heregulin or a variant thereof; and wherein a chemotherapeutic drug (such as doxorubicin) is intercalated into the double-stranded oligonucleotide. In some embodiments, the cell-targeting segment binds a cancer cell, such as a HER3+ cancer cell. In some embodiments, the cancer cell is a chemotherapeutic drug resistant cancer cell. In some embodiments, the double-stranded oligonucleotide is between about 20 and about 50 bases in length. In some embodiments, the molar ratio of the chemotherapeutic drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a composition comprising nanoparticles comprising a carrier polypeptide and a double-stranded DNA oligonucleotide, the carrier polypeptide comprises a cell-targeting segment, a cell-penetrating segment, and an oligonucleotide-binding segment; wherein the molar ratio of the carrier polypeptide to the double-stranded oligonucleotide in the nanoparticles is about 4:1; wherein the carrier polypeptide is HerPBK10, and wherein doxorubicin is intercalated into the double-stranded oligonucleotide. In some embodiments, the cell-targeting segment binds a cancer cell, such as a HER3+ cancer cell. In some embodiments, the cancer cell is a chemotherapeutic drug resistant cancer cell. In some embodiments, the double-stranded oligonucleotide is between about 20 and about 50 bases in length. In some embodiments, the molar ratio of the chemotherapeutic drug to the double-stranded oligonucleotide is between about 1:1 to about 60:1 (such as about 10:1 or about 40:1). In some embodiments, the average size of the nanoparticles in the composition is no greater than about 50 nm.

### Production of Nanoparticles

The nanoparticles described herein can be produced by combining a plurality of carrier polypeptides with a plurality of double-stranded oligonucleotides. In some embodiments, the carrier polypeptides, the double-stranded oligonucleotides, and optionally a small-molecule drug are incubated to form the nanoparticles. In some embodiments, the oligonucleotides are pre-incubated with a small molecule drug prior to being combined with the carrier polypeptides. Upon combining the carrier polypeptide and the double-stranded oligonucleotides, the nanoparticles spontaneously assemble.

In some embodiments, there is provided a method of making a nanoparticle composition comprising combining a carrier polypeptide and a double-stranded oligonucleotide at a molar ratio of less than about 6:1, thereby forming a plurality of nanoparticles; wherein the carrier polypeptide comprises a cell-targeting segment, a cell-penetrating segment, and an oligonucleotide-binding segment. In some embodiments, the method further comprises combining the double-stranded oligonucleotide and a small-molecule drug prior to combining the carrier polypeptide and the double-stranded oligonucleotide.

In some embodiments, single-stranded, complementary (or partially complementary) oligonucleotides are annealed to form double-stranded oligonucleotides. Annealing of the oligonucleotides can occur, for example, by combining approximately equimolar amounts of each single-stranded oligonucleotide, heating the oligonucleotides (for example, to about 90° C. or higher), and cooling the mixture (for example, at about room temperature).

The small molecule drug (such as the chemotherapeutic agent, for example, doxorubicin) can be bound to the double-stranded oligonucleotide by combining the small molecule drug and double-stranded oligonucleotide. In some embodiments, the small molecule drug and the double-stranded oligonucleotide are combined at a molar ratio of about 60:1 or less, about 50:1 or less, about 40:1 or less, about 30:1 or less, about 20:1 or less, about 10:1 or less, about 5:1 or less, about 4:1 or less, about 3:1 or less, about 2:1 or less, or about 1:1 or less. In some embodiments, the small molecule drug and the double-stranded oligonucleotide are combined at a molar ratio between about 1:1 and about 60:1, such as between about 1:1 and about 10:1, between about 5:1 and about 20:1, between about 10:1 and about 30:1, between about 20:1 and about 40:1, between about 30:1 and about 50:1, or between about 40:1 and about 60:1, at about 1:1, at about 1:10, at about 1:20, at about 1:30, at about 1:40, at about 1:50, or at about 1:60. Once the small molecule drug and the double-stranded oligonucleotide are combined, the small molecule drug binds to the double-stranded oligonucleotide, for example by intercalating into the double-stranded oligonucleotide.

The double-stranded oligonucleotide, which is optionally bound by the small molecule drug, is combined with the carrier polypeptide to form the nanoparticles. In some embodiments, the carrier peptide and the double-stranded oligonucleotide are combined at a molar ratio of 1 less than about 6:1 (for example, about 4:1 to less than about 6:1, such as about 4:1 to about 4.5:1, about 4.5:1 to about 5:1, about 5:1 to about 5.5:1, about 5.5:1 to less than about 6:1, about 4:1, about 4.5:1, about 5:1, or about 5.5). In some embodiments, the carrier polypeptide and the double-stranded oligonucleotide are incubated at about 4° C. to about 22° C., such as between about 4° C. and about 15° C., or between about 4° C. and about 10° C. In some embodiments, the carrier polypeptide and the double-stranded oligonucleotide incubate for less than about 30 minutes, about 30 minutes or more, about 1 hour or more, or about 2 hours or more. After combining the carrier polypeptide with the double-stranded oligonucleotide, the nanoparticles spontaneously form.

In some embodiments, excess oligonucleotide, small molecule drug, or carrier polypeptide are removed from the composition comprising the nanoparticles. For example, in some embodiments, the nanoparticle composition is subjected to a purification step, such as size exclusion chromatography. In some embodiments, the unbound components are separated from the nanoparticles by ultracentrifugation. For example, in some embodiments, the composition is added to a centrifugal filter with a molecular weight cutoff of about 100 kD or less, about 80 kD or less, about 70 kD or less, about 60 kD or less, about 50 kD or less, about 40 kD or less, about 30 kD or less, or about 20 kD or less.

Optionally, the resulting nanoparticle composition is subjected to buffer exchange, for example by dialysis, ultracentrifugation, or tangential flow filtration. In some embodiments, the nanoparticles are concentrated, for example by ultracentrifugation.

In some embodiments, there is provided a method of making a nanoparticle composition comprising combining a carrier polypeptide and a double-stranded oligonucleotide (such as DNA) at a molar ratio of less than about 6:1 (such as about 4:1 to less than about 6:1, or about 4:1), thereby forming a plurality of nanoparticles; wherein the carrier polypeptide comprises a cell-targeting segment, a cell-penetrating segment, and an oligonucleotide-binding segment. In some embodiments, the cell-penetrating segment comprises (and, in some embodiments, is) a penton base polypeptide or a variant thereof. In some embodiments, the oligonucleotide binding domain is positively charged (such as decalysine). In some embodiments, the cell-targeting domain comprises (and, in some embodiments, is) heregulin or a variant thereof. In some embodiments, the cell-penetrating segment is a penton base polypeptide or a variant thereof, the oligonucleotide binding domain is positively charged (such as decalysine), and the cell-targeting domain is heregulin or a variant thereof. In some embodiments, the average size of the resulting nanoparticles in the composition is no greater than about 50 nm.

In some embodiments, there is provided a method of making a nanoparticle composition comprising combining a double-stranded oligonucleotide (such as DNA) and a small-molecule drug (such as a chemotherapeutic drug, for example doxorubicin); and combining a carrier polypeptide and the double-stranded oligonucleotide at a molar ratio of less than about 6:1 (such as about 4:1 to less than about 6:1, or about 4:1), thereby forming a plurality of nanoparticles; wherein the carrier polypeptide comprises a cell-targeting segment, a cell-penetrating segment, and an oligonucleotide-binding segment. In some embodiments, the cell-penetrating segment comprises (and, in some embodiments, is) a penton base polypeptide or a variant thereof. In some embodiments, the oligonucleotide binding domain is positively charged (such as decalysine). In some embodiments, the cell-targeting domain comprises (and, in some embodiments, is) heregulin or a variant thereof. In some embodiments, the cell-penetrating segment is a penton base polypeptide or a variant thereof, the oligonucleotide binding domain is positively charged (such as decalysine), and the cell-targeting domain is heregulin or a variant thereof. In some embodiments, the average size of the resulting nanoparticles in the composition is no greater than about 50 nm.

In some embodiments, there is provided a method of making a nanoparticle composition comprising combining a double-stranded oligonucleotide (such as DNA) and a small-molecule drug (such as a chemotherapeutic drug, for example doxorubicin); combining a carrier polypeptide and the double-stranded oligonucleotide at a molar ratio of less than about 6:1 (such as about 4:1 to less than about 6:1, or about 4:1), thereby forming a plurality of nanoparticles; and separating unbound carrier polypeptide or double-stranded oligonucleotide from the plurality of nanoparticles; wherein the carrier polypeptide comprises a cell-targeting segment, a cell-penetrating segment, and an oligonucleotide-binding segment. In some embodiments, the cell-penetrating segment comprises (and, in some embodiments, is) a penton base polypeptide or a variant thereof. In some embodiments, the oligonucleotide binding domain is positively charged (such as decalysine). In some embodiments, the cell-targeting domain comprises (and, in some embodiments, is) heregulin or a variant thereof. In some embodiments, the cell-penetrating segment is a penton base polypeptide or a variant thereof, the oligonucleotide binding domain is positively charged (such as decalysine), and the cell-targeting domain is heregulin or a variant thereof. In some embodiments, the average size of the resulting nanoparticles in the composition is no greater than about 50 nm.

In some embodiments, there is provided a method of making a nanoparticle composition comprising combining a double-stranded oligonucleotide (such as DNA) and a small-molecule drug (such as a chemotherapeutic drug, for example doxorubicin); separating unbound small-molecule drug from the double-stranded oligonucleotide; combining a carrier polypeptide and the double-stranded oligonucleotide at a molar ratio of less than about 6:1 (such as about 4:1 to less than about 6:1, or about 4:1), thereby forming a plurality of nanoparticles; and separating unbound carrier polypeptide or double-stranded oligonucleotide from the plurality of nanoparticles; wherein the carrier polypeptide comprises a cell-targeting segment, a cell-penetrating segment, and an oligonucleotide-binding segment. In some embodiments, the cell-penetrating segment comprises (and, in some embodiments, is) a penton base polypeptide or a variant thereof. In some embodiments, the oligonucleotide binding domain is positively charged (such as decalysine). In some embodiments, the cell-targeting domain comprises (and, in some embodiments, is) heregulin or a variant thereof. In some embodiments, the cell-penetrating segment is a penton base polypeptide or a variant thereof, the oligonucleotide binding domain is positively charged (such as decalysine), and the cell-targeting domain is heregulin or a variant thereof. In some embodiments, the average size of the resulting nanoparticles in the composition is no greater than about 50 nm.

### Cancer Treatments

Nanoparticle compositions can be useful for the treatment of cancer in a subject by administering an effective amount of a composition comprising the nanoparticles to the subject, thereby killing the cancer cells. The cell-targeting segment of the carrier polypeptide can target a molecule on the surface of a cancer cell, thereby delivering a chemotherapeutic agent (which can be bound to the double-stranded oligonucleotide) to the cancer cells. In some embodiments, the cancer is metastatic. In some embodiments, the cancer is a chemotherapeutic drug-resistant cancer, as further described herein.

In one aspect, there is provided a method of killing a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide bound to the oligonucleotide-binding segment; and a chemotherapeutic drug bound to the double-stranded oligonucleotide; wherein the molar ratio of the carrier polypeptide to the double-stranded oligonucleotide in the plurality of nanoparticles is less than about 6:1.

In another aspect, there is provided a method of treating a subject with a cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide bound to the oligonucleotide-binding segment; and a chemotherapeutic drug bound to the double-stranded oligonucleotide; wherein the carrier wherein the molar ratio of the carrier polypeptide to the double-stranded oligonucleotide in the nanoparticle composition is less than about 6:1.

In another aspect, there is provided a method of delivering a chemotherapeutic agent to a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide bound to the oligonucleotide-binding segment; and a chemotherapeutic drug bound to the double-stranded oligonucleotide; wherein the carrier wherein the molar ratio of the carrier polypeptide to the double-stranded oligonucleotide in the plurality of nanoparticles is less than about 6:1.

In some embodiments, the cancer is a HER3+ cancer. A Her cell-targeting segment, for example, can bind HER3 present on the surface of the HER3+ cancer cells to target the nanoparticles to the cancer cells.

In some embodiments, an effective amount of a composition comprising the nanoparticles is administered to subject to treat a glioma, breast cancer, ovarian cancer, or prostate cancer. In some embodiments, any one of these cancers is HER3+. In some embodiments, the cancer is negative for one or more of the progesterone receptor (PR), the estrogen receptor (ER), or HER2 (e.g., PR⁻, ER⁻, HER2⁻, PR⁻/ER⁻, etc.). In some embodiments, the cancer is triple negative breast cancer.

In some embodiments, a composition comprising the nanoparticles is used to kill a cancer cell, such as a glioma cell, a breast cancer cell, an ovarian cancer cell, or a prostate cancer cell. In some embodiments, any one of these cancer cells is HER3+. In some embodiments, the cancer cell is negative for one or more of the progesterone receptor (PR), the estrogen receptor (ER), or HER2 (e.g., PR⁻, ER⁻, HER2⁻, PR⁻/ER⁻, etc.). In some embodiments, the cancer cell is a triple negative breast cancer cell.

In some embodiments, the nanoparticles described herein are more potent than liposomal doxorubicin (or “LipoDox,” for example the composition sold under the brand name Doxil®). An exemplary nanoparticles comprises a carrier polypeptide and a double-stranded oligonucleotide at an average molar ratio between 4:1 and less than about 6:1 (carrier polypeptide to double-stranded oligonucleotide), and comprise a small molecule drug (such as doxorubicin) at an average molar ratio of about 1:1 to about 60:1 (small molecule drug to oligonucleotide). For example, in some embodiments, the nanoparticles comprise a carrier polypeptide and a double-stranded oligonucleotide at an average molar ratio of about 4:1 (carrier polypeptide to double-stranded oligonucleotide), and a small molecule drug (such as doxorubicin) at an average molar ratio of about 10:1 (small molecule drug to oligonucleotide). In another example, in some embodiments, the nanoparticles comprise a carrier polypeptide and a double-stranded oligonucleotide at an average molar ratio of about 4:1 (carrier polypeptide to double-stranded oligonucleotide), and a small molecule drug (such as doxorubicin) at an average molar ratio of about 40:1 (small molecule drug to oligonucleotide).

In some embodiments, the cancer cell proliferates in the presence of the drug. In some embodiments, a culture of cancer cells does not shrink in the presence of the drug. In some embodiments, the cancer cell is not killed in the presence of the drug. In some embodiments, the relative cell survival of the cancer cell line is about 0.7 or higher (such as about 0.8 or higher, or about 0.9 or higher) at a dosage and length of time that results in a non-drug resistant cell line of the same cancer cell type having a relative cell survival of about 0.5 or lower (such as about 0.4 or lower, about 0.3 or lower, or about 0.2 or lower).

In some embodiments, the cancer or cancer cell to be treated or killed is non-responsive to a chemotherapeutic drug, such as a small-molecule drug or an antibody. In some embodiments, the cancer or cancer cell to be treated or killed is non-responsive to a liposomal formulation of a chemotherapeutic drug, such as a liposomal anthracycline. In some embodiments, the cancer or cancer cell to be treated or killed is non-responsive to a HER2+ antibody chemotherapeutic agent, lapatinib, or an anthracycline. In some embodiments, the cancer or cancer cell to be treated or killed is non-responsive to doxorubicin (which may be in the form of nanoparticle doxorubicin, such as liposomal doxorubicin, or a non-nanoparticle formulation of doxorubicin). In some embodiments, the cancer or cancer cell to be treated or killed is non-responsive to lapatinib. In some embodiments, the cancer or cancer cell to be treated or killed is non-responsive to trastuzumab and/or pertuzumab.

In some embodiments, the describe method comprises identifying a subject with a cancer that is non-responsive to a chemotherapeutic agent, and administering an effective amount of a composition comprising nanoparticles as described herein. In some embodiments, the cancer or cancer cell is non-responsive to an anti-HER2 treatment (such as an anti-HER2 antibody or a small-molecule inhibitor of HER2 (e.g., lapatinib). In some embodiments, the cancer or cancer cell is non-responsive to an anti-HER2 antibody treatment, such as trastuzumab and/or pertuzumab. In some embodiments, the cancer or cancer cell is non-responsive to doxorubicin (such as a liposomal formulation of doxorubicin, or a non-nanoparticle formulation of doxorubicin).

In some embodiments, the nanoparticles described herein are more effective at killing HER3+ cancer cells, such as MDA-MB-435 cells, than liposomal doxorubicin. In some embodiments, the nanoparticles described herein have an IC50 for killing HER3+ cancer cells (such as MDA-MB-435 cells) of less than about 10 μM, such as less than about 5 μM. In some embodiments, the nanoparticles described herein have an IC50 for killing HER3+ cancer cells (such as MDA-MB-435 cells) of between about 2 μM and about 10 μM.

In some embodiments, the nanoparticles described herein are more effective at killing breast cancer cells, such as BT474 breast cancer cells or JIMT1 breast cancer cells, than liposomal doxorubicin. In some embodiments, the nanoparticles described herein have an IC50 for killing breast cancer cells (such as BT474 breast cancer cells or JIMT1 breast cancer cells) of less than about 10 μM, such as less than about 5 μM, less than about 1 μM, or less than about 0.5 μM. In some embodiments, the nanoparticles described herein have an IC50 for killing breast cancer cells (such as BT474 breast cancer cells or JIMT1 breast cancer cells) of between about 0.1 μM and about 10 μM, such as between about 0.5 μM and about 10 μM, or between about 0.5 UM and about 1 μM.

In some embodiments, the nanoparticles described herein are more effective at killing triple negative breast cancer cells, such as 4T1 triple negative mammary cancer cells, than liposomal doxorubicin. In some embodiments, the nanoparticles described herein have an IC50 for killing triple negative breast cancer cells (such as 4T1 triple negative mammary cancer cells) of less than about 10 μM, such as less than about 5 μM, less than about 1 μM, or less than about 0.5 μM. In some embodiments, the nanoparticles described herein have an IC50 for killing triple negative breast cancer cells (such as 4T1 triple negative mammary cancer cells) of between about 0.1 μM and about 10 μM, such as between about 0.5 UM and about 10 μM, or between about 0.5 μM and about 1 μM.

In some embodiments, the nanoparticles described herein are more effective at killing glioma cells, such as U251 glioma cells, than liposomal doxorubicin. In some embodiments, the nanoparticles described herein have an IC50 for killing glioma cells (such as U251 glioma cells) of less than about 10 UM, such as less than about 5 μM, less than about 1 μM, or less than about 0.5 μM. In some embodiments, the nanoparticles described herein have an IC50 for killing glioma cells (such as U251 glioma cells) of between about 0.1 μM and about 10 μM, such as between about 0.5 μM and about 10 μM, or between about 0.5 μM and about 1 μM.

In some embodiments, the nanoparticles described herein are more effective at killing ovarian cancer cells, such as SKOV3 ovarian cancer cells, than liposomal doxorubicin. In some embodiments, the nanoparticles described herein have an IC50 for killing ovarian cancer cells (such as SKOV3 ovarian cancer cells) of less than about 10 μM, such as less than about 5 μM, or less than about 1 μM. In some embodiments, the nanoparticles described herein have an IC50 for killing ovarian cancer cells (such as SKOV3 ovarian cancer cells) of between about 0.1 μM and about 10 μM, such as between about 0.5 μM and about 10 μM, or between about 0.5 μM and about 1 μM.

In some embodiments, the nanoparticles described herein are more effective at killing prostate cancer cells, such as LNCaP-GFP prostate cancer cells, than liposomal doxorubicin. In some embodiments, the nanoparticles described herein have an IC50 for killing prostate cancer cells (such as LNCaP-GFP prostate cancer cells) of less than about 10 μM, such as less than about 5 μM, less than about 1 μM, or less than about 0.5 μM. In some embodiments, the nanoparticles described herein have an IC50 for killing prostate cancer cells (such as LNCaP-GFP prostate cancer cells) of between about 0.1 μM and about 10 μM, such as between about 0.5 UM and about 10 μM, or between about 0.5 μM and about 1 μM.

In some embodiments, the nanoparticles described herein are more effective at killing metastatic cancer cells, such as bone-metastatic prostate cancer cells (for example, RANKL human bone-metastatic prostate cancer cells), than liposomal doxorubicin. In some embodiments, the nanoparticles described herein have an IC50 for killing metastatic cancer cells, such as bone-metastatic prostate cancer cells (for example, RANKL human bone-metastatic prostate cancer cells) of less than about 10 UM, such as less than about 5 μM, less than about 1 μM, or less than about 0.5 μM. In some embodiments, the nanoparticles described herein have an IC50 for killing metastatic cancer cells, such as bone-metastatic prostate cancer cells (for example, RANKL human bone-metastatic prostate cancer cells) of between about 0.1 μM and about 10 μM, such as between about 0.5 μM and about 10 μM, or between about 0.5 μM and about 1 μM.

In some embodiments, the method of treating a subject with cancer further comprises a secondary therapy, such as radiation therapy or surgery. Thus, in some embodiments, the composition comprising the nanoparticles described herein is administered to a subject with cancer as a neoadjuvant therapy and/or an adjuvant therapy. For example, in some embodiments, trastuzumab and/or pertuzumab are used as an adjuvant to an anticancer therapy comprising administering the nanoparticle composition described herein.

In some embodiments, the subject has not undergone chemotherapy or radiation therapy prior to administration of the nanoparticles described herein. In some embodiments, the subject has undergone chemotherapy or radiation therapy.

In some embodiments, the nanoparticle composition described herein is administered to a subject. In some embodiments, the nanoparticle composition is administered to a subject for in vivo delivery to targeted cells. Generally, dosages and routes of administration of the nanoparticle composition are determined according to the size and condition of the subject, according to standard pharmaceutical practice. In some embodiments, the nanoparticle composition is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, or intraspinally. In some embodiments, the composition is administered to a subject intravenously.

In some embodiments, the dosage of the nanoparticle composition is a single dose or a repeated dose. In some embodiments, the doses are given to a subject once per day, twice per day, three times per day, or four or more times per day. In some embodiments, about 1 or more (such as about 2 or more, about 3 or more, about 4 or more, about 5 or more, about 6 or more, or about 7 or more) doses are given in a week. In some embodiments, the composition is administered weekly, once every 2 weeks, once every 3 weeks, once every 4 weeks, weekly for two weeks out of 3 weeks, or weekly for 3 weeks out of 4 weeks. In some embodiments, multiple doses are given over the course of days, weeks, months, or years. In some embodiments, a course of treatment is about 1 or more doses (such as about 2 or more does, about 3 or more doses, about 4 or more doses, about 5 or more doses, about 7 or more doses, about 10 or more doses, about 15 or more doses, about 25 or more doses, about 40 or more doses, about 50 or more doses, or about 100 or more doses).

In some embodiments, an administered dose of the nanoparticle composition is about 200 mg/m² or lower of the small molecule drug (such as doxorubicin), about 150 mg/m² or lower of the small molecule drug (such as doxorubicin), about 100 mg/m² or lower of the small molecule drug (such as doxorubicin), about 80 mg/m² or lower of the small molecule drug (such as doxorubicin), about 70 mg/m² or lower of the small molecule drug (such as doxorubicin), about 60 mg/m² or lower of the small molecule drug (such as doxorubicin), about 50 mg/m² or lower of the small molecule drug (such as doxorubicin), about 40 mg/m² or lower of the small molecule drug (such as doxorubicin), about 30 mg/m² or lower of the small molecule drug (such as doxorubicin), about 20 mg/m² or lower of the small molecule drug (such as doxorubicin), about 15 mg/m² or lower of the small molecule drug (such as doxorubicin), about 10 mg/m² or lower of the small molecule drug (such as doxorubicin), about 5 mg/m² or lower of the small molecule drug (such as doxorubicin), or about 1 mg/m² or lower of the small molecule drug (such as doxorubicin). In some embodiments, the administered dose of the nanoparticle composition is less than the dose of liposomal doxorubicin for approximately the same therapeutic effect. In some embodiments, the administered dose of the nanoparticle composition provides an increased therapeutic effect relative to the therapeutic effect of about the same dose of liposomal doxorubicin.

In some embodiments, there is provided a method of treating a subject with a cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticle composition is less than about 6:1 (such as 4:1 to less than about 6:1, or about 4:1). In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1). In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticle composition is less than about 6:1 (such as 4:1 to less than about 6:1, or about 4:1); and wherein the cell-penetrating segment comprises (and, in some embodiments, is) a penton base polypeptide or a variant thereof. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1); and wherein the cell-penetrating segment comprises (and, in some embodiments, is) a penton base polypeptide or a variant thereof. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticle composition is less than about 6:1 (such as 4:1 to less than about 6:1, or about 4:1); wherein the cell-penetrating segment comprises (and, in some embodiments, is) a penton base polypeptide or a variant thereof; and wherein the oligonucleotide-binding segment is positively charged. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1); wherein the cell-penetrating segment comprises (and, in some embodiments, is) a penton base polypeptide or a variant thereof; and wherein the oligonucleotide-binding segment is positively charged. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticle composition is less than about 6:1 (such as 4:1 to less than about 6:1, or about 4:1); wherein the cell-penetrating segment comprises (and, in some embodiments, is) a penton base polypeptide or a variant thereof; wherein the oligonucleotide-binding segment is positively charged; and wherein the cell-targeting segment comprises (and, in some embodiments, is) heregulin or a variant thereof. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1); wherein the cell-penetrating segment comprises (and, in some embodiments, is) a penton base polypeptide or a variant thereof; wherein the oligonucleotide-binding segment is positively charged; and wherein the cell-targeting segment comprises (and, in some embodiments, is) heregulin or a variant thereof. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticle composition is less than about 6:1 (such as 4:1 to less than about 6:1, or about 4:1); wherein the cell-penetrating segment comprises (and, in some embodiments, is) a penton base polypeptide or a variant thereof; wherein the oligonucleotide-binding segment comprises (and, in some embodiments, is) decalysine; and wherein the cell-targeting segment comprises (and, in some embodiments, is) heregulin or a variant thereof. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles less than about 6:1 (such as about 4:1 to less than about 6:1, about 5:1, or about 4:1); wherein the cell-penetrating segment comprises (and, in some embodiments, is) a penton base polypeptide or a variant thereof; wherein the oligonucleotide-binding segment comprises (and, in some embodiments, is) decalysine; and wherein the cell-targeting segment comprises (and, in some embodiments, is) heregulin or a variant thereof. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticle composition is less than about 6:1 (such as 4:1 to less than about 6:1, or about 4:1); wherein the cell-penetrating segment comprises (and, in some embodiments, is) a penton base polypeptide or a variant thereof; wherein the oligonucleotide-binding segment comprises (and, in some embodiments, is) decalysine; and wherein the cell-targeting segment comprises (and, in some embodiments, is) heregulin or a variant thereof; and wherein a chemotherapeutic drug (such as doxorubicin) is intercalated into the double-stranded oligonucleotide. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1); wherein the cell-penetrating segment comprises (and, in some embodiments, is) a penton base polypeptide or a variant thereof; wherein the oligonucleotide-binding segment comprises (and, in some embodiments, is) decalysine; wherein the cell-targeting segment comprises (and, in some embodiments, is) heregulin or a variant thereof; and wherein a chemotherapeutic drug (such as doxorubicin) is intercalated into the double-stranded oligonucleotide. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a cancer, comprising administering to the subject a nanoparticle composition comprising nanoparticles, the nanoparticles comprising a carrier polypeptide and a double-stranded DNA oligonucleotide, the carrier polypeptide comprises a cell-targeting segment, a cell-penetrating segment, and an oligonucleotide-binding segment; wherein the molar ratio of the carrier polypeptide to the double-stranded oligonucleotide in the nanoparticles is about 4:1; wherein the carrier polypeptide is HerPBK10, and wherein doxorubicin is intercalated into the double-stranded oligonucleotide. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of killing a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the plurality of nanoparticles is less than about 6:1 (such as 4:1 to less than about 6:1, or about 4:1). In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1). In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the plurality of nanoparticles is less than about 6:1 (such as 4:1 to less than about 6:1, or about 4:1); and wherein the cell-penetrating segment comprises (and, in some embodiments, is) a penton base polypeptide or a variant thereof. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1); and wherein the cell-penetrating segment comprises (and, in some embodiments, is) a penton base polypeptide or a variant thereof. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the plurality of nanoparticles is less than about 6:1 (such as 4:1 to less than about 6:1, or about 4:1); wherein the cell-penetrating segment comprises (and, in some embodiments, is) a penton base polypeptide or a variant thereof; and wherein the oligonucleotide-binding segment is positively charged. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1); wherein the cell-penetrating segment comprises (and, in some embodiments, is) a penton base polypeptide or a variant thereof; and wherein the oligonucleotide-binding segment is positively charged. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the plurality of nanoparticles is less than about 6:1 (such as 4:1 to less than about 6:1, or about 4:1); wherein the cell-penetrating segment comprises (and, in some embodiments, is) a penton base polypeptide or a variant thereof; wherein the oligonucleotide-binding segment is positively charged; and wherein the cell-targeting segment comprises (and, in some embodiments, is) heregulin or a variant thereof. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1); wherein the cell-penetrating segment comprises (and, in some embodiments, is) a penton base polypeptide or a variant thereof; wherein the oligonucleotide-binding segment is positively charged; and wherein the cell-targeting segment comprises (and, in some embodiments, is) heregulin or a variant thereof. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the plurality of nanoparticles is less than about 6:1 (such as 4:1 to less than about 6:1, or about 4:1); wherein the cell-penetrating segment comprises (and, in some embodiments, is) a penton base polypeptide or a variant thereof; wherein the oligonucleotide-binding segment comprises (and, in some embodiments, is) decalysine; and wherein the cell-targeting segment comprises (and, in some embodiments, is) heregulin or a variant thereof. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1); wherein the cell-penetrating segment comprises (and, in some embodiments, is) a penton base polypeptide or a variant thereof; wherein the oligonucleotide-binding segment comprises (and, in some embodiments, is) decalysine; and wherein the cell-targeting segment comprises (and, in some embodiments, is) heregulin or a variant thereof. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the plurality of nanoparticles is less than about 6:1 (such as 4:1 to less than about 6:1, or about 4:1); wherein the cell-penetrating segment comprises (and, in some embodiments, is) a penton base polypeptide or a variant thereof; wherein the oligonucleotide-binding segment comprises (and, in some embodiments, is) decalysine; and wherein the cell-targeting segment comprises (and, in some embodiments, is) heregulin or a variant thereof; and wherein a chemotherapeutic drug (such as doxorubicin) is intercalated into the double-stranded oligonucleotide. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1); wherein the cell-penetrating segment comprises (and, in some embodiments, is) a penton base polypeptide or a variant thereof; wherein the oligonucleotide-binding segment comprises (and, in some embodiments, is) decalysine; wherein the cell-targeting segment comprises (and, in some embodiments, is) heregulin or a variant thereof; and wherein a chemotherapeutic drug (such as doxorubicin) is intercalated into the double-stranded oligonucleotide. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a cancer cell comprising contacting the cancer cell with a plurality of nanoparticles, the nanoparticles comprising a carrier polypeptide and a double-stranded DNA oligonucleotide, the carrier polypeptide comprises a cell-targeting segment, a cell-penetrating segment, and an oligonucleotide-binding segment; wherein the molar ratio of the carrier polypeptide to the double-stranded oligonucleotide in the nanoparticles is about 4:1; wherein the carrier polypeptide is HerPBK10, and wherein doxorubicin is intercalated into the double-stranded oligonucleotide. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of delivering a chemotherapeutic agent to a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the plurality of nanoparticles is less than about 6:1 (such as 4:1 to less than about 6:1, or about 4:1). In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of delivering a chemotherapeutic agent to a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1). In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of delivering a chemotherapeutic agent to a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the plurality of nanoparticles is less than about 6:1 (such as 4:1 to less than about 6:1, or about 4:1); and wherein the cell-penetrating segment comprises (and, in some embodiments, is) a penton base polypeptide or a variant thereof. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of delivering a chemotherapeutic agent to a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1); and wherein the cell-penetrating segment comprises (and, in some embodiments, is) a penton base polypeptide or a variant thereof. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of delivering a chemotherapeutic agent to a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the plurality of nanoparticles is less than about 6:1 (such as 4:1 to less than about 6:1, or about 4:1); wherein the cell-penetrating segment comprises (and, in some embodiments, is) a penton base polypeptide or a variant thereof; and wherein the oligonucleotide-binding segment is positively charged. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of delivering a chemotherapeutic agent to a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1); wherein the cell-penetrating segment comprises (and, in some embodiments, is) a penton base polypeptide or a variant thereof; and wherein the oligonucleotide-binding segment is positively charged. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of delivering a chemotherapeutic agent to a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the plurality of nanoparticles is less than about 6:1 (such as 4:1 to less than about 6:1, or about 4:1); wherein the cell-penetrating segment comprises (and, in some embodiments, is) a penton base polypeptide or a variant thereof; wherein the oligonucleotide-binding segment is positively charged; and wherein the cell-targeting segment comprises (and, in some embodiments, is) heregulin or a variant thereof. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of delivering a chemotherapeutic agent to a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1); wherein the cell-penetrating segment comprises (and, in some embodiments, is) a penton base polypeptide or a variant thereof; wherein the oligonucleotide-binding segment is positively charged; and wherein the cell-targeting segment comprises (and, in some embodiments, is) heregulin or a variant thereof. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of delivering a chemotherapeutic agent to a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the plurality of nanoparticles is less than about 6:1 (such as 4:1 to less than about 6:1, or about 4:1); wherein the cell-penetrating segment comprises (and, in some embodiments, is) a penton base polypeptide or a variant thereof; wherein the oligonucleotide-binding segment comprises (and, in some embodiments, is) decalysine; and wherein the cell-targeting segment comprises (and, in some embodiments, is) heregulin or a variant thereof. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of delivering a chemotherapeutic agent to a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1); wherein the cell-penetrating segment comprises (and, in some embodiments, is) a penton base polypeptide or a variant thereof; wherein the oligonucleotide-binding segment comprises (and, in some embodiments, is) decalysine; and wherein the cell-targeting segment comprises (and, in some embodiments, is) heregulin or a variant thereof. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of delivering a chemotherapeutic agent to a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the plurality of nanoparticles is less than about 6:1 (such as 4:1 to less than about 6:1, or about 4:1); wherein the cell-penetrating segment comprises (and, in some embodiments, is) a penton base polypeptide or a variant thereof; wherein the oligonucleotide-binding segment comprises (and, in some embodiments, is) decalysine; and wherein the cell-targeting segment comprises (and, in some embodiments, is) heregulin or a variant thereof; and wherein a chemotherapeutic drug (such as doxorubicin) is intercalated into the double-stranded oligonucleotide. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of delivering a chemotherapeutic agent to a cancer cell comprising contacting the cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the molar ratio of the carrier polypeptide to the double-stranded oligonucleotide in the nanoparticles is less than about 6:1 (such as about 4:1 to less than about 6:1, about 5:1, or about 4:1); wherein the cell-penetrating segment comprises (and, in some embodiments, is) a penton base polypeptide or a variant thereof; wherein the oligonucleotide-binding segment comprises (and, in some embodiments, is) decalysine; wherein the cell-targeting segment comprises (and, in some embodiments, is) heregulin or a variant thereof; and wherein a chemotherapeutic drug (such as doxorubicin) is intercalated into the double-stranded oligonucleotide. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

n some embodiments, there is provided a method of delivering a chemotherapeutic agent to a cancer cell comprising contacting the cancer cell with a plurality of nanoparticles, the nanoparticles comprising a carrier polypeptide and a double-stranded DNA oligonucleotide, the carrier polypeptide comprises a cell-targeting segment, a cell-penetrating segment, and an oligonucleotide-binding segment; wherein the molar ratio of the carrier polypeptide to the double-stranded oligonucleotide in the nanoparticles is about 4:1; wherein the carrier polypeptide is HerPBK10, and wherein doxorubicin is intercalated into the double-stranded oligonucleotide. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

### Methods of Treating Drug Resistant Cancer

Nanoparticle compositions can also be useful for killing a chemotherapeutic drug-resistant cancer and the treatment of a subject with a chemotherapeutic drug-resistant cancer. In some embodiments, there is provided a method of killing a chemotherapeutic drug-resistant cancer cell comprising contacting the chemotherapeutic drug-resistant cancer cell with a plurality of nanoparticles, the nanoparticles comprising a carrier polypeptide comprising a cell-targeting segment, a cell-penetrating segment, and an oligonucleotide-binding segment; a double-stranded oligonucleotide bound to the oligonucleotide-binding segment; and a chemotherapeutic drug bound to the double-stranded oligonucleotide.

In some embodiments, there is provided a method of treating a subject with a chemotherapeutic drug-resistant cancer, comprising administering to the subject a composition comprising a plurality of nanoparticles, the nanoparticles comprising a carrier polypeptide comprising a cell-targeting segment, a cell-penetrating segment, and an oligonucleotide-binding segment; a double-stranded oligonucleotide bound to the oligonucleotide-binding segment; and a chemotherapeutic drug bound to the double-stranded oligonucleotide.

In some embodiments, there is provided a method of delivering a chemotherapeutic agent to a chemotherapeutic drug-resistant cancer cell comprising contacting the chemotherapeutic drug-resistant cancer cell with a plurality of nanoparticles, the nanoparticles comprising a carrier polypeptide comprising a cell-targeting segment, a cell-penetrating segment, and an oligonucleotide-binding segment; a double-stranded oligonucleotide bound to the oligonucleotide-binding segment; and a chemotherapeutic drug bound to the double-stranded oligonucleotide.

The methods described herein are also useful for treating subjects who have progressed on the prior therapy with a drug (such as a chemotherapeutic agent) at the time of treatment. For example, the subject has progressed within any of about 1, 2, 3, 4, 5, 6, 7, 8, 9, 10, 11, or 12 months upon treatment with the prior therapy. In some embodiments, the subject with cancer is initially responsive to the treatment with the prior therapy, but develops a recurrent cancer after about any of about 6, 7, 8, 9, 10, 11, 12, 24, or 36 months upon the cessation of the prior therapy.

Although the description below describes subjects that are resistant to a prior therapy (such as a doxorubicin-based therapy) as exemplary embodiments, it is understood that the description herein also applies to subjects who have progressed on the prior therapy, subjects that are unsuitable to continue with the prior therapy (for example due to failure to respond and/or due to toxicity), subjects that have recurrent cancer after the prior therapy, subjects that are non-responsive to the prior therapy, subjects that exhibit a less desirable degree of responsiveness and/or subjects that exhibit enhanced responsiveness. The methods described herein include all second-line therapies for treating cancers that involve the administration of a nanoparticle composition described herein.

The nanoparticles can kill the chemotherapeutic drug-resistant cancer cell either in vivo or in vitro. The nanoparticles can also kill the drug-resistant cancer cell in vitro, for example by mixing a composition comprising the nanoparticles with drug-resistant cancer cells. The cell-targeting segment of the carrier polypeptide can bind to a molecule present on the surface of the cancer cell. For example, in some embodiments, the drug-resistant cancer cell is a HER3+ cell, and the cell-targeting segment binds to HER3. The nanoparticles can also be used to kill a chemotherapeutic drug-resistant cancer in vivo, for example by administering a composition comprising the nanoparticles to a subject with a drug-resistant cancer. In some embodiments, the nanoparticles are used to treat a subject with a drug resistant cancer, for example by administering an effective amount of a composition comprising the nanoparticles to the subject.

In some embodiments, the drug-resistant cancer is resistant to an antibody. For example, in some embodiments, the drug-resistant cancer is resistant to an anti-HER2 antibody, such as trastuzumab (also known under the brand name, Herceptin®). In some embodiments, the drug-resistant cancer is resistant to pertuzumab. In many cases, trastuzumab or pertuzumab loses its effectivity in certain cancer types during the course of therapy. This frequently occurs during the treatment of breast cancer. However, the described nanoparticles are still able to target the trastuzumab resistant cancer cells or pertuzumab resistant cancer cells, and thus are effective in killing the cancer cells or treating patients with a trastuzumab-resistant cancer or pertuzumab-resistant cancer.

In some embodiments, the nanoparticles described herein are effective for treating cancer which is resistant to liposomal doxorubicin. In some embodiments, the nanoparticles are effective for killing a HER2 antibody (such as trastuzumab or pertuzumab) resistant cancer. In some embodiments, the nanoparticles are more effective at killing HER2 antibody (such as trastuzumab or pertuzumab) resistant breast cancer cells, such as trastuzumab-resistant BT474-TR breast cancer cells, than liposomal doxorubicin. In some embodiments, the nanoparticles described herein have an IC50 for killing HER2 antibody (such as trastuzumab) resistant breast cancer cells (such as trastuzumab-resistant BT474-TR breast cancer cells) of less than about 10 μM, such as less than about 5 μM, less than about 1 μM, or less than about 0.5 μM. In some embodiments, the nanoparticles described herein have an IC50 for killing HER2 antibody (such as trastuzumab) resistant breast cancer cells (such as trastuzumab-resistant BT474-TR breast cancer cells) of between about 0.01 μM and about 10 μM, such as between about 0.1 μM and about 1 μM, or between about 0.5 μM and about 1 μM.

In some embodiments, the drug-resistant cancer is resistant to a small molecule chemotherapeutic agent, such as an anthracycline (for example, doxorubicin, also known under the brand name Adriamycin®) or a tyrosine-kinase inhibitor (such as lapatinib). In some embodiments, the drug-resistant cancer is resistant to LipoDox.

The nanoparticles described herein increase cell death of a doxorubicin-resistant cell line at an equivalent amount of doxorubicin as liposomal doxorubicin, which indicates that the nanoparticles are more effective than liposomal doxorubicin in treating patients exhibiting resistance to doxorubicin. In some embodiments, the nanoparticles described herein are more effective at killing cancer cells that are resistant to a small molecule chemotherapeutic agent, such as doxorubicin, (for example, A2780-ADR Adriamycin-resistant human ovarian cancer cells), than liposomal doxorubicin.

In some embodiments, there is provided a method of treating a subject with a chemotherapeutic drug-resistant cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the chemotherapeutic drug-resistant cancer is resistant to a HER2+ antibody (such as trastuzumab or pertuzumab), an anthracycline (such as doxorubicin), or a tyrosine-kinase inhibitor (such as lapatinib). In some embodiments, the molar ratio of the carrier polypeptide to the double-stranded oligonucleotide in the nanoparticle composition is less than about 6:1 (such as 4:1 to less than about 6:1, or about 4:1). In some embodiments, the molar ratio of the carrier polypeptide to the double-stranded oligonucleotide in the nanoparticles is less than about 6:1 (such as about 4:1 to less than about 6:1, about 5:1, or about 4:1). In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a chemotherapeutic drug-resistant cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; and wherein the cell-penetrating segment comprises (and, in some embodiments, is) a penton base polypeptide or a variant thereof. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the chemotherapeutic drug-resistant cancer is resistant to a HER2+ antibody (such as trastuzumab or pertuzumab), an anthracycline (such as doxorubicin), or a tyrosine-kinase inhibitor (such as lapatinib). In some embodiments, the molar ratio of the carrier polypeptide to the double-stranded oligonucleotide in the nanoparticle composition is less than about 6:1 (such as 4:1 to less than about 6:1, or about 4:1). In some embodiments, the molar ratio of the carrier polypeptide to the double-stranded oligonucleotide in the nanoparticles is less than about 6:1 (such as about 4:1 to less than about 6:1, about 5:1, or about 4:1). In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a chemotherapeutic drug-resistant cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the cell-penetrating segment comprises (and, in some embodiments, is) a penton base polypeptide or a variant thereof; and wherein the oligonucleotide-binding segment is positively charged. In some embodiments, the cancer is a HER3+ cancer. In some embodiments, the chemotherapeutic drug-resistant cancer is resistant to a HER2+ antibody (such as trastuzumab or pertuzumab), an anthracycline (such as doxorubicin), or a tyrosine-kinase inhibitor (such as lapatinib). In some embodiments, the molar ratio of the carrier polypeptide to the double-stranded oligonucleotide in the nanoparticle composition is less than about 6:1 (such as 4:1 to less than about 6:1, or about 4:1). In some embodiments, the molar ratio of the carrier polypeptide to the double-stranded oligonucleotide in the nanoparticles is less than about 6:1 (such as about 4:1 to less than about 6:1, about 5:1, or about 4:1). In some embodiments, the cancer is breast cancer (such as triple negative breast cancer), glioma, ovarian cancer, or a prostate cancer, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a chemotherapeutic drug-resistant cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the cell-penetrating segment comprises (and, in some embodiments, is) a penton base polypeptide or a variant thereof; wherein the oligonucleotide-binding segment is positively charged; and wherein the cell-targeting segment comprises (and, in some embodiments, is) heregulin or a variant thereof. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the chemotherapeutic drug-resistant cancer is resistant to a HER2+ antibody (such as trastuzumab or pertuzumab), an anthracycline (such as doxorubicin), or a tyrosine-kinase inhibitor (such as lapatinib). In some embodiments, the molar ratio of the carrier polypeptide to the double-stranded oligonucleotide in the nanoparticle composition is less than about 6:1 (such as 4:1 to less than about 6:1, or about 4:1). In some embodiments, the molar ratio of the carrier polypeptide to the double-stranded oligonucleotide in the nanoparticles is less than about 6:1 (such as about 4:1 to less than about 6:1, about 5:1, or about 4:1). In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a chemotherapeutic drug-resistant cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the cell-penetrating segment comprises (and, in some embodiments, is) a penton base polypeptide or a variant thereof; wherein the oligonucleotide-binding segment comprises (and, in some embodiments, is) decalysine; and wherein the cell-targeting segment comprises (and, in some embodiments, is) heregulin or a variant thereof. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the chemotherapeutic drug-resistant cancer is resistant to a HER2+ antibody (such as trastuzumab or pertuzumab), an anthracycline (such as doxorubicin), or a tyrosine-kinase inhibitor (such as lapatinib). In some embodiments, the molar ratio of the carrier polypeptide to the double-stranded oligonucleotide in the nanoparticle composition is less than about 6:1 (such as 4:1 to less than about 6:1, or about 4:1). In some embodiments, the molar ratio of the carrier polypeptide to the double-stranded oligonucleotide in the nanoparticles is less than about 6:1 (such as about 4:1 to less than about 6:1, about 5:1, or about 4:1). In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a chemotherapeutic drug-resistant cancer, comprising administering to the subject a nanoparticle composition comprising nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the cell-penetrating segment comprises (and, in some embodiments, is) a penton base polypeptide or a variant thereof; wherein the oligonucleotide-binding segment comprises (and, in some embodiments, is) decalysine; and wherein the cell-targeting segment comprises (and, in some embodiments, is) heregulin or a variant thereof; and wherein a chemotherapeutic drug (such as doxorubicin) is intercalated into the double-stranded oligonucleotide. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the chemotherapeutic drug-resistant cancer is resistant to a HER2+ antibody (such as trastuzumab or pertuzumab), an anthracycline (such as doxorubicin), or a tyrosine-kinase inhibitor (such as lapatinib). In some embodiments, the molar ratio of the carrier polypeptide to the double-stranded oligonucleotide in the nanoparticle composition is less than about 6:1 (such as 4:1 to less than about 6:1, or about 4:1). In some embodiments, the molar ratio of the carrier polypeptide to the double-stranded oligonucleotide in the nanoparticles is less than about 6:1 (such as about 4:1 to less than about 6:1, about 5:1, or about 4:1). In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm.

In some embodiments, there is provided a method of treating a subject with a chemotherapeutic drug-resistant cancer, comprising administering to the subject a nanoparticle composition comprising nanoparticles, the nanoparticles comprising a carrier polypeptide and a double-stranded DNA oligonucleotide, the carrier polypeptide comprises a cell-targeting segment, a cell-penetrating segment, and an oligonucleotide-binding segment; wherein the molar ratio of the carrier polypeptide to the double-stranded oligonucleotide in the nanoparticles is about 4:1; wherein the carrier polypeptide is HerPBK10, and wherein doxorubicin is intercalated into the double-stranded oligonucleotide. In some embodiments, the cancer is a HER3+ cancer. In some embodiments, the cancer is breast cancer (such as triple negative breast cancer), glioma, ovarian cancer, or a prostate cancer, any one of which may be HER3+. In some embodiments, the chemotherapeutic drug-resistant cancer is resistant to a HER2+ antibody (such as trastuzumab or pertuzumab), an anthracycline (such as doxorubicin), or a tyrosine-kinase inhibitor (such as lapatinib). In some embodiments, the molar ratio of the carrier polypeptide to the double-stranded oligonucleotide in the nanoparticle composition is less than about 6:1 (such as 4:1 to less than about 6:1, or about 4:1). In some embodiments, the molar ratio of the carrier polypeptide to the double-stranded oligonucleotide in the nanoparticles is less than about 6:1 (such as about 4:1 to less than about 6:1, about 5:1, or about 4:1). In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a chemotherapeutic drug-resistant cancer cell comprising contacting the chemotherapeutic drug-resistant cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the chemotherapeutic drug-resistant cancer is resistant to a HER2+ antibody (such as trastuzumab or pertuzumab), an anthracycline (such as doxorubicin), or a tyrosine-kinase inhibitor (such as lapatinib). In some embodiments, the molar ratio of the carrier polypeptide to the double-stranded oligonucleotide in the nanoparticle composition is less than about 6:1 (such as 4:1 to less than about 6:1, or about 4:1). In some embodiments, the molar ratio of the carrier polypeptide to the double-stranded oligonucleotide in the nanoparticles is less than about 6:1 (such as about 4:1 to less than about 6:1, about 5:1, or about 4:1). In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles in the composition is no greater than about 50 nm. In some embodiments, the average size of the nanoparticles is no greater than about 50 nm.

In some embodiments, there is provided a method of killing a chemotherapeutic drug-resistant cancer cell comprising contacting the chemotherapeutic drug-resistant cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; and wherein the cell-penetrating segment comprises (and, in some embodiments, is) a penton base polypeptide or a variant thereof. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the chemotherapeutic drug-resistant cancer is resistant to a HER2+ antibody (such as trastuzumab or pertuzumab), an anthracycline (such as doxorubicin), or a tyrosine-kinase inhibitor (such as lapatinib). In some embodiments, the molar ratio of the carrier polypeptide to the double-stranded oligonucleotide in the nanoparticle composition is less than about 6:1 (such as 4:1 to less than about 6:1, or about 4:1). In some embodiments, the molar ratio of the carrier polypeptide to the double-stranded oligonucleotide in the nanoparticles is less than about 6:1 (such as about 4:1 to less than about 6:1, about 5:1, or about 4:1). In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a chemotherapeutic drug-resistant cancer cell comprising contacting the chemotherapeutic drug-resistant cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the cell-penetrating segment comprises (and, in some embodiments, is) a penton base polypeptide or a variant thereof; and wherein the oligonucleotide-binding segment is positively charged. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the chemotherapeutic drug-resistant cancer is resistant to a HER2+ antibody (such as trastuzumab or pertuzumab), an anthracycline (such as doxorubicin), or a tyrosine-kinase inhibitor (such as lapatinib). In some embodiments, the molar ratio of the carrier polypeptide to the double-stranded oligonucleotide in the nanoparticle composition is less than about 6:1 (such as 4:1 to less than about 6:1, or about 4:1). In some embodiments, the molar ratio of the carrier polypeptide to the double-stranded oligonucleotide in the nanoparticles is less than about 6:1 (such as about 4:1 to less than about 6:1, about 5:1, or about 4:1). In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a chemotherapeutic drug-resistant cancer cell comprising contacting the chemotherapeutic drug-resistant cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the cell-penetrating segment comprises (and, in some embodiments, is) a penton base polypeptide or a variant thereof; wherein the oligonucleotide-binding segment is positively charged; and wherein the cell-targeting segment comprises (and, in some embodiments, is) heregulin or a variant thereof. In some embodiments, the cancer cell is a HER3+ cancer cell. In some embodiments, the chemotherapeutic drug-resistant cancer is resistant to a HER2+ antibody (such as trastuzumab or pertuzumab), an anthracycline (such as doxorubicin), or a tyrosine-kinase inhibitor (such as lapatinib). In some embodiments, the molar ratio of the carrier polypeptide to the double-stranded oligonucleotide in the nanoparticle composition is less than about 6:1 (such as 4:1 to less than about 6:1, or about 4:1). In some embodiments, the molar ratio of the carrier polypeptide to the double-stranded oligonucleotide in the nanoparticles is less than about 6:1 (such as about 4:1 to less than about 6:1, about 5:1, or about 4:1). In some embodiments, the cancer cell is a breast cancer cell (such as a triple negative breast cancer cell), a glial cancer cell, an ovarian cancer cell, or a prostate cancer cell, any one of which may be HER3+. In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a chemotherapeutic drug-resistant cancer cell comprising contacting the chemotherapeutic drug-resistant cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the cell-penetrating segment comprises (and, in some embodiments, is) a penton base polypeptide or a variant thereof; wherein the oligonucleotide-binding segment comprises (and, in some embodiments, is) decalysine; and wherein the cell-targeting segment comprises (and, in some embodiments, is) heregulin or a variant thereof. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the chemotherapeutic drug-resistant cancer is resistant to a HER2+ antibody (such as trastuzumab or pertuzumab), an anthracycline (such as doxorubicin), or a tyrosine-kinase inhibitor (such as lapatinib). In some embodiments, the molar ratio of the carrier polypeptide to the double-stranded oligonucleotide in the nanoparticle composition is less than about 6:1 (such as 4:1 to less than about 6:1, or about 4:1). In some embodiments, the molar ratio of the carrier polypeptide to the double-stranded oligonucleotide in the nanoparticles is less than about 6:1 (such as about 4:1 to less than about 6:1, about 5:1, or about 4:1). In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a chemotherapeutic drug-resistant cancer cell comprising contacting the chemotherapeutic drug-resistant cancer cell with a plurality of nanoparticles, the nanoparticles comprising a carrier polypeptide comprising a cell-targeting segment, a cell-penetrating segment, and an oligonucleotide-binding segment; a double-stranded oligonucleotide (such as DNA) bound to the oligonucleotide-binding segment; and a chemotherapeutic drug (such as an anthracycline, for example doxorubicin) bound to the double-stranded oligonucleotide; wherein the cell-penetrating segment comprises (and, in some embodiments, is) a penton base polypeptide or a variant thereof; wherein the oligonucleotide-binding segment comprises (and, in some embodiments, is) decalysine; and wherein the cell-targeting segment comprises (and, in some embodiments, is) heregulin or a variant thereof; and wherein a chemotherapeutic drug (such as doxorubicin) is intercalated into the double-stranded oligonucleotide. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the chemotherapeutic drug-resistant cancer is resistant to a HER2+ antibody (such as trastuzumab or pertuzumab), an anthracycline (such as doxorubicin), or a tyrosine-kinase inhibitor (such as lapatinib). In some embodiments, the molar ratio of the carrier polypeptide to the double-stranded oligonucleotide in the nanoparticle composition is less than about 6:1 (such as 4:1 to less than about 6:1, or about 4:1). In some embodiments, the molar ratio of the carrier polypeptide to the double-stranded oligonucleotide in the nanoparticles is less than about 6:1 (such as about 4:1 to less than about 6:1, about 5:1, or about 4:1). In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

In some embodiments, there is provided a method of killing a chemotherapeutic drug-resistant cancer cell comprising contacting the chemotherapeutic drug-resistant cancer cell with a plurality of nanoparticles, the nanoparticles comprising a carrier polypeptide and a double-stranded DNA oligonucleotide, the carrier polypeptide comprises a cell-targeting segment, a cell-penetrating segment, and an oligonucleotide-binding segment; wherein the molar ratio of the carrier polypeptide to the double-stranded oligonucleotide in the nanoparticles is about 4:1; wherein the carrier polypeptide is HerPBK10, and wherein doxorubicin is intercalated into the double-stranded oligonucleotide. In some embodiments, the cancer cell is a HER3+ cancer cell. In some embodiments, the cancer cell is a breast cancer cell (such as a triple negative breast cancer cell), a glial cancer cell, an ovarian cancer cell, or a prostate cancer cell, any one of which may be HER3+. In some embodiments, the chemotherapeutic drug-resistant cancer is resistant to a HER2+ antibody (such as trastuzumab or pertuzumab), an anthracycline (such as doxorubicin), or a tyrosine-kinase inhibitor (such as lapatinib). In some embodiments, the molar ratio of the carrier polypeptide to the double-stranded oligonucleotide in the nanoparticle composition is less than about 6:1 (such as 4:1 to less than about 6:1, or about 4:1). In some embodiments, the molar ratio of the carrier polypeptide to the double-stranded oligonucleotide in the nanoparticles is less than about 6:1 (such as about 4:1 to less than about 6:1, about 5:1, or about 4:1). In some embodiments, the double-stranded oligonucleotide is between about 20 and about 50 bases in length. In some embodiments, the molar ratio of the small molecule drug to the double-stranded oligonucleotide is between about 1:1 to about 60:1 (such as about 10:1 or about 40:1). In some embodiments, the average size of the nanoparticles is no greater than about 50 nm.

### Pharmaceutical Compositions

In some embodiments, the compositions described herein are formulated as pharmaceutical compositions comprising a plurality of nanoparticles described herein and a pharmaceutically acceptable excipient.

In some embodiments, the pharmaceutical composition is a solid, such as a powder. The powder can be formed, for example, by lyophilizing the nanoparticles in solution. The powder can be reconstituted, for example by mixing the powder with an aqueous liquid (e.g., water or a buffer). In some embodiments, the pharmaceutical composition is a liquid, for example nanoparticles suspended in an aqueous solution (such as physiological saline or Ringer's solution). In some embodiments, the pharmaceutical composition comprises a pharmaceutically-acceptable excipient, for example a filler, binder, coating, preservative, lubricant, flavoring agent, sweetening agent, coloring agent, a solvent, a buffering agent, a chelating agent, or stabilizer.

Examples of pharmaceutically-acceptable fillers include cellulose, dibasic calcium phosphate, calcium carbonate, microcrystalline cellulose, sucrose, lactose, glucose, mannitol, sorbitol, maltol, pregelatinized starch, corn starch, or potato starch. Examples of pharmaceutically-acceptable binders include polyvinylpyrrolidone, starch, lactose, xylitol, sorbitol, maltitol, gelatin, sucrose, polyethylene glycol, methyl cellulose, or cellulose. Examples of pharmaceutically-acceptable coatings include hydroxypropyl methylcellulose (HPMC), shellac, corn protein zein, or gelatin. Examples of pharmaceutically-acceptable disintegrants include polyvinylpyrrolidone, carboxymethyl cellulose, or sodium starch glycolate. Examples of pharmaceutically-acceptable lubricants include polyethylene glycol, magnesium stearate, or stearic acid. Examples of pharmaceutically-acceptable preservatives include methyl parabens, ethyl parabens, propyl paraben, benzoic acid, or sorbic acid. Examples of pharmaceutically-acceptable sweetening agents include sucrose, saccharine, aspartame, or sorbitol. Examples of pharmaceutically-acceptable buffering agents include carbonates, citrates, gluconates, acetates, phosphates, or tartrates.

### Articles of Manufacture and Kits

Also provided are articles of manufacture comprising the compositions described herein in suitable packaging. Suitable packaging for compositions described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The present invention also provides kits comprising compositions (or articles of manufacture) described herein and may further comprise instruction(s) on methods of using the composition, such as uses described herein. The kits described herein may further include other materials desirable from a commercial and user standpoint, including other buffers, diluents, filters, needles, syringes, and package inserts with instructions for performing any methods described herein.

### EXAMPLES

The examples provided herein are included for illustrative purposes only and are not intended to limit the scope of the invention.

### Example 1: Nanoparticle Assembly

Nanoparticles comprising a carrier polypeptide, a double-stranded DNA oligonucleotide, and doxorubicin (referred to as “HerDox” particles) were assembled using the following methods.

Single stranded, complementary DNA oligonucleotides (Eurofins Operon; sequences were as follows LLAA-5: 5′-CGCCTGAGCAACGCGGCGGGCATCCGCAAG-3′ (SEQ ID NO:5) and LLAA-3: 3′-GCGGACTCGTTGCGCCGCCCGTAGGCGTTC-5′) (SEQ ID NO:6)) were annealed by incubating equal molar ratios of each oligonucleotide in boiling water for 5 minutes. The oligonucleotides were then cooled at room temperature for 30 minutes.

The double-stranded, annealed, DNA oligonucleotides were then incubated with doxorubicin HCl at a molar ratio of 1:40 DNA:Dox at room temperature for 30 minutes.

The doxorubicin-bound double-stranded DNA oligonucleotides were then incubated with a carrier polypeptide (“HerPBK10”) comprising a Her cell-targeting segment, a PB cell-penetrating segment, and a decalysine (“K10”) oligonucleotide binding segment at a molar ratio of 4:1 HerPBK10:DNA-doxorubicin (thus a molar ratio of 4:1:40 HerPBK10:DNA:doxorubicin) in HEPES Buffered Saline (HBS). The mixture of carrier polypeptide and doxorubicin-bound double stranded DNA oligonucleotides was rocked for 2 hours on ice, thereby forming the HerDox particles.

The resulting nanoparticles were then subjected to ultracentrifugation. Specifically, 12 mL of sterile HBS was added to a 50 kD cut-off Centrifugal Filter (Amicon Ultra-15) that had been pre-incubated in sterile, 10% glycerol for 24 hours. The HerDox mixtures were added to the cold HBS in the centrifugal filer. The filter tubes were then spun for 10-20 minutes at 2500 RPM (5000×g) in a Beckman J6-HC centrifuge until the final volume was between 200 μL and 500 μL. The concentrated HerDox was then transferred to a 1.7 mL microfuge tube.

Empty nanoparticles were prepared by incubating HerPBK10 with the double-stranded DNA oligonucleotide (no doxorubicin) as described for HerDox nanoparticles, but without incubating the double-stranded oligonucleotide with the doxorubicin. Similar mixtures can be made using molar ratios of HerPBK10:DNA of 2:1, 3:1, 4:1, 5:1, and 6:1, and/or with a molar ratio of dsDNA:doxorubicin of about 1:10 or about 1:40.

Treatment doses for the Examples described below reflect the doxorubicin concentration in HerDox, which was determined by extrapolating the measured absorbance (A480) against a Dox absorbance calibration curve (SpectraMax MA; Molecular Devices, CA, USA). Normalization of treatment concentrations for the Empty nanoparticles (HerPBK10-DNA) was based on HerPBK10 content relative to HerDox.

### Example 2: Nanoparticle Size

HerPBK10 carrier polypeptides were combined with doxorubicin-intercalated double stranded DNA (1:10 molar ratio dsDNA:doxorubicin) at a molar ratio of 2:1, 3:1, 4:1, 5:1, or 6:1. The mixture was then subjected to dynamic light scattering (DLS) to determine the diameter of the resulting nanoparticles. Solutions of HerPBK10 (no oligonucleotides or doxorubicin) and doxorubicin-intercalated double stranded DNA (no HerPBK10) were also measured by DLS. Results are presented in FIG. 2. As seen in FIG. 2, nanoparticles of about 35 nm formed when molar ratios of 4:1, 5:1 and 6:1 (HerPBK10:dsDNA) were combined.

### Example 3: CryoEM of Nanoparticles

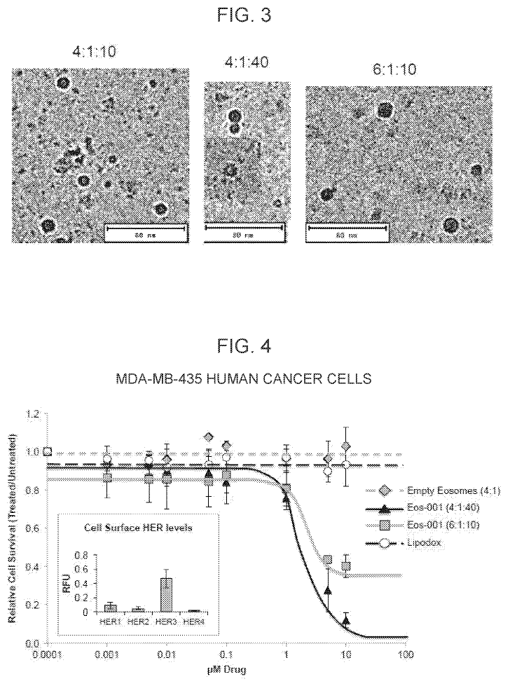

Doxorubicin was combined with double stranded DNA, followed by combining the mixture with HerPBK10 carrier polypeptides at molar ratios of 4:1:10, 4:1:40, or 6:1:10 (HerPBK10:dsDNA:doxorubicin). The mixture was then imaged using cryoEM, and is presented in FIG. 3. As show in FIG. 3, all three mixtures produce nanoparticle of similar size and morphology.

### Example 4: Use of Nanoparticles to Kill Cancer Cells and Chemotherapeutic Drug Resistant Cancer Cells

Nanoparticles with either no doxorubicin (4:1 molar ratio of HerPBK10:dsDNA, referred to in this example as “Empty Eosomes”), nanoparticles with a 4:1:40 molar ratio of HerPBK10:dsDNA:doxorubicin (referred to in this Example as Eos-001 (4:1:40)), or nanoparticles with a 6:1:10 molar ratio of HerPBK10:dsDNA:doxorubicin (referred to in this Example as Eos-001 (6:1:10)) were compared to LipoDox for its ability to kill various types of cancer cells.

Various doses of nanoparticles were incubated with either MDA-MB-435 (human cancer) cells, BT474 (human breast cancer) cells, BT474-R (trastuzumab-resistant human breast cancer) cells, JIMT1 (human breast cancer cells from a patient naturally resistant to trastuzumab), U251 (human glioma) cells, A2780-ADR (doxorubicin-resistant human ovarian cancer) cells, 4T1 (triple-negative mouse mammary cancer) cells, SKOV3 (human ovarian cancer) cells, LNCaP-GFP (human prostate cancer) cells, RANKL (human bone-metastatic prostate cancer cells), or BT-549 (triple-negative human breast cancer) cells.

### Cells

SKBR3 and MDA-MB-435 cells were obtained from ATCC. BT-474 cells and JIMT1 cells were obtained from Cedars-Sinai Medical Center. All cells except JIMT1 were maintained at 37° C. in complete media DMEM (Dulbecco's modified Eagle's medium), 10% heat inactivated fetal bovine serum and 100 U/mL penicillin/100 μg/mL streptomycin at 5% CO₂. JIMT1 cells were maintained in RPMI (Roswell Park Memorial Institute Media), 10% heat inactivated fetal bovine serum, 100 U/mL penicillin/100 μg/ml streptomycin and 1 mM Sodium Pyruvate at 5% CO₂.

### Cell Surface ELISA Assay

The relative amounts of HER1, HER2, HER3, or HER4 present on the surface of the various cell lines was determined using and ELISA assay. Cells were plated at 8,000 or 10,000 cells per well in black walled, clear bottomed 96-well plates and allowed to grow for 48 hours at 37° C. and 5% CO₂. Cells were washed once with PBS+ (1× Phosphate Buffered Saline (PBS) with 1% MgCl₂ and 1% CaCl₂)), fixed with 4% Paraformaldehyde (PFA) in PBS for 12 minutes with rocking and then blocked with 3% Bovine Serum Albumin (BSA) in PBS for 3 hours with gentle agitation. The block solution was removed and the indicated primary antibodies (HER1, HER2, HER3, or HER4 antibodies) were added to the plate at 1:500 dilution, diluted in 3% BSA in PBS and incubated overnight at 4° C. while rocking. The plate was washed 3 times with PBS with 5 minutes incubation with gentle agitation between washes. The appropriate secondary antibody was added at 1:1000 dilution, diluted in 3% BSA in PBS and the plate was incubated for 1 hour at room temperature with gentle agitation. Cells were washed 3 times with PBS with 5 minutes incubation with gentle agitation between washes and then once with diH₂O. All liquid was removed from the wells and 100 μL of tetramethylbenzidine (TMB) substrate (eBioscience) was added to each well and the plate was developed for ˜30 minutes in the dark with gentle agitation. Once sufficient blue color had developed, reactions were quantified by measuring absorbance at 650 nm using a plate reader. 100 μL of 1N HCl was then added to each well of the plate to stop the reaction and the plate was read again at 450 nm. The TMB/HCl solution was removed and the plate was washed twice with 1×PBS. 50 μL of 0.1% Crystal Violet in 100% ethanol was added to each well. The plate was incubated in the dark for 30 minutes with gentle rocking. The plate was thoroughly washed with PBS and then 100 μL of 95% ethanol was added to each well to release the crystal violet from any cells. The plate was then read at 590 nm. The crystal violet approximates the number of cells per well and enables the normalization of each assay and comparison across plates.

### Cell Viability Assay

Relative cell survival after exposure to the described compositions was measured using a cell viability assay. 15,000, 10,000, or 8,000 cells per well were plated in black-walled, clear-bottom, 96-well plates. 48 hours later, the media was aspirated and replaced with complete media and the indicated concentrations of Empty Eosomes, Eos-001 (4:1:40), Eos-001 (6:1:10), or LipoDox at a total volume of 40 μL. Plates were rocked for 4 hours at 37° C. and 5% CO₂ and then 60 μL of complete media was added to each well to bring the total volume to 100 μL and the incubation was continued, without rocking, for 44 hours at 37° C. and 5% CO₂. At the conclusion of the incubation, relative cell viability was determined via MTS assay (Promega) according to manufacturer's instructions. Specifically, the media was removed from the wells and 100 μL of fresh complete media was added to each well. 20 μl of the prepared MTS reagent was added to each well. The plate was then incubated with rocking at 37° C. and 5% CO₂ and readings were taken of the plate at 1, 2, and 3 hours at 490 nm using a spectrophotometer. The results are shown in terms of the following ratio: number of cells that survived in the treatment group divided by the number of cells that survived in the untreated group. Thus, cell survival of 1.0 indicates that the treated cells and the untreated cells survived to the same extent, whereas a ratio of 0.2 means that as compared with the untreated cell group, only 20% of the treated cells survived.

### Results

The results are shown in the Figures. Throughout the Figures, “Empty Eosomes (4:1)” refer to nanoparticles comprising the HerPBK10 carrier polypeptide and double-stranded DNA oligonucleotide at a 4:1 molar ratio of HerPBK10:dsDNA, but no doxorubicin; “Empty Eosomes (6:1)” refer to nanoparticles comprising the HerPBK10 carrier polypeptide and double-stranded DNA oligonucleotide at a 6:1 molar ratio of HerPBK10:dsDNA, but no doxorubicin; “Eos-001 (4:1:40)” refers to the nanoparticles comprising the HerPBK10 carrier polypeptide, double-stranded DNA oligonucleotide, and doxorubicin at a 4:1:40 molar ratio of HerPBK10:dsDNA:doxorubicin; “Eos-001 (6:1:10)” refers to the nanoparticles comprising the HerPBK10 carrier polypeptide, double-stranded DNA oligonucleotide, and doxorubicin at a 6:1:10 molar ratio of HerPBK10:dsDNA:doxorubicin; and “LipoDox” refers to commercially available liposomal doxorubicin. Subset in each Figure is the relevant amounts of HER1, HER2, HER3, or HER4 present on the surface of each cell type.

Referring to FIG. 4, it is shown that LipoDox and Empty Eosomes (4:1) have no noticeable effect on the survival of MDA-MB-435 cells. In contrast Eos-001 (6:1:10) particles demonstrate a significant decrease of MDA-MB-435 cell survival at concentrations over 1 μM doxorubicin. Eos-001 (4:1:40) particles demonstrate an even more significant decrease in MDA-MB-435 cell survival at concentrations over 1 μM doxorubicin, with less than 20% of cells surviving at a concentration of about 10 μM doxorubicin. The inset graph compares the cell surface levels of various HER receptors, showing that HER3 is the most prevalent receptor.

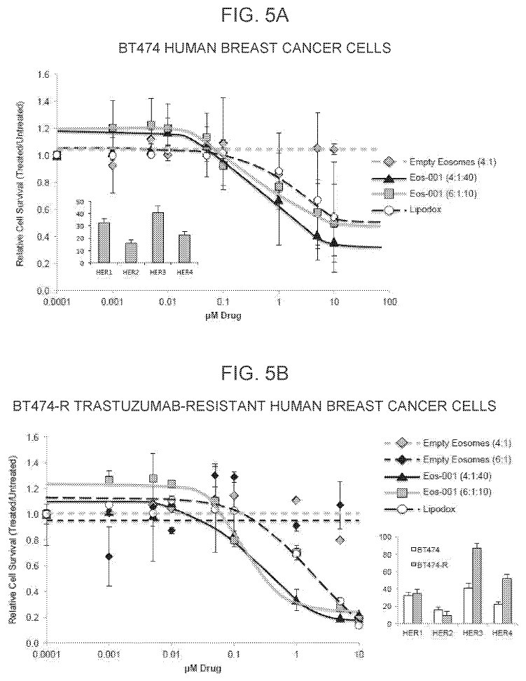

Referring to FIG. 5A, it is shown that Empty Eosomes (4:1) have no noticeable effect on the survival of BT474 human breast cancer cells. Each of LipoDox, Eos-001 (6:1:10), and Eos-001 (4:1:40) reduced the survival of the BT474 cells, although Eos-001 (4:1:40) reduced the survival of the BT474 cells most significantly. Referring to FIG. 5B, it is shown that neither Empty Eosomes (4:1) or Empty Eosomes (6:1) had noticeable effect on the survival of the BT474-R trastuzumab resistant human breast cancer cells. LipoDox did decrease cell survival partially after administration of about 1 μM doxorubicin. However, administration of Eos-001 (4:1:40) or Eos-001 (6:1:10) results in an even greater decrease in relative cell survival at approximately the same concentration.

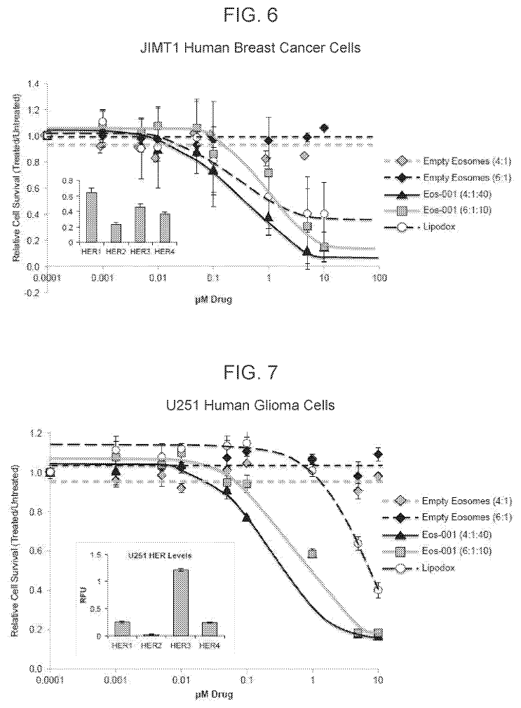

Referring to FIG. 6, it is shown that LipoDox's efficacy on JIMT1 cells plateaus at about 40% survival despite increasing the drug concentration by a factor of approximately 10. However, Eos-001 (4:1:40) and Eos-001 (6:1:10) reduces the survival of JIMT1 cells at lower concentrations while achieving a survival rate of less than 10%. The inset graph compares the cell surface levels of various HER receptors.

Referring to FIG. 7, it is shown that LipoDox reduces the survival of U251 human glioma cells at significantly greater concentrations of doxorubicin than Eos-001 (4:1:40) or Eos-001 (6:1:10). Both Eos-001 (4:1:40) or Eos-001 (6:1:10) result in less than about 20% survival at concentrations of about 10 μM doxorubicin. In contrast, administration of LipoDox results in approximately 40% cell survival at the same concentration. The inset graph compares the cell surface levels of various HER receptors, showing that HER3 is the most prevalent receptor.

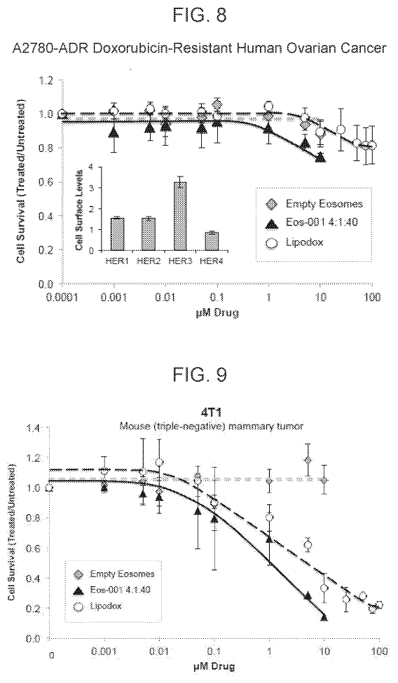

Referring to FIG. 8, it is shown that Eos-001 (4:1:40) has a significantly greater effect in decreasing cell survival of A2780-ADR doxorubicin-resistant human ovarian cancer cells than LipoDox.

Referring to FIG. 9, it is shown that Eos-001 (4:1:40) has a significantly greater effect in decreasing cell survival of 4T1 triple-negative mouse mammary cancer cells than LipoDox.

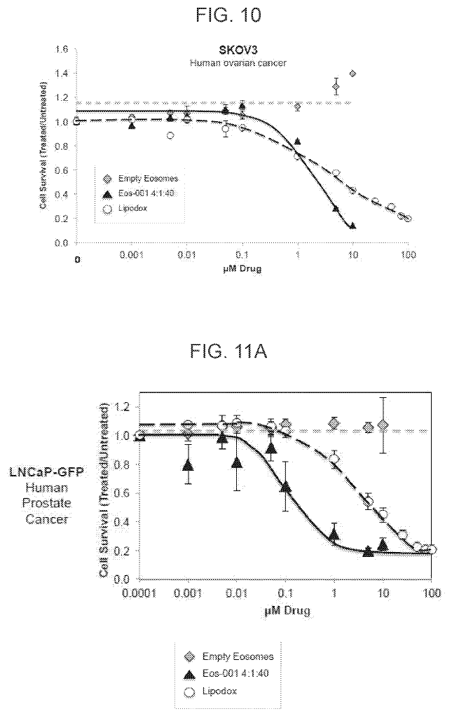

Referring to FIG. 10, it is shown that Eos-001 (4:1:40) has a significantly greater effect in decreasing cell survival of SKOV3 human ovarian cancer cells than LipoDox.

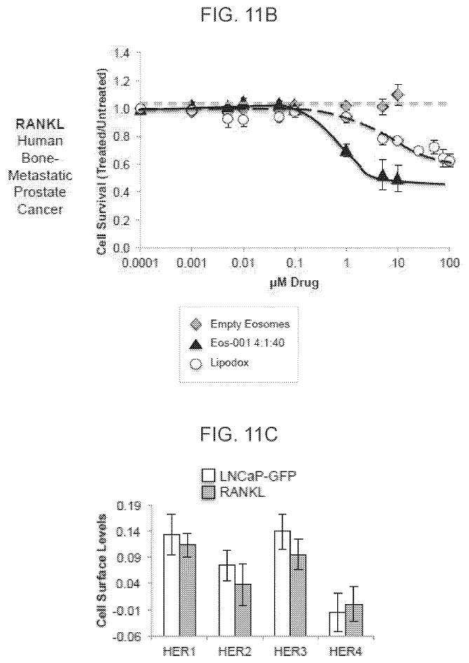

Referring to FIG. 11A, it is shown that Eos-001 (4:1:40) has a significantly greater effect in decreasing cell survival of LNCaP-GFP human prostate cancer cells than LipoDox. Referring to FIG. 11B, it is shown that Eos-001 (4:1:40) has a significantly greater effect in decreasing cell survival of RANKL human bone-metastatic prostate cancer cells than LipoDox. FIG. 11C shows the relative expression of HER1, HER2, HER3, and HER4 in LNCaP-GFP and RANKL cells.

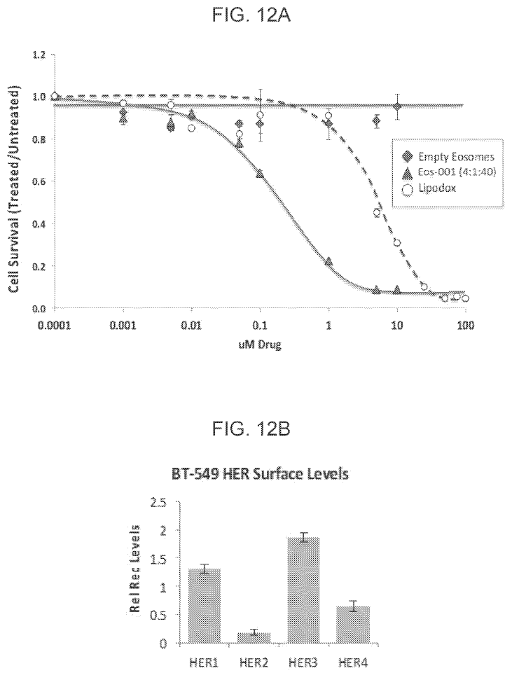

FIG. 12A shows that Eos-001 (4:1:40) has a significantly greater effect in decreasing the survival of BT549 human triple-negative breast cancer cells than LipoDox. FIG. 12B shows the relative expression of HER1, HER 2, HER3, and HER4 in BT549 cells.

### Example 5: Comparing Nanoparticles to Anti-HER2 Antibody Treatments in Killing Chemotherapeutic Drug Resistant Cancer Cells

BT474 (human breast cancer) cells, BT474-TR (trastuzumab-resistant human breast cancer) cells, SKBR3 (human breast cancer) cells, and SKBR3-TR (trastuzumab resistant breast cancer) cells were incubated with various concentrations of Eos-001, trastuzumab, or combination trastuzumab and pertuzumab. The concentration of Eos-001 is reported in μM doxorubicin, and the concentration of trastuzumab or pertuzumab is reported in μM antibody. The cells per well were plated in black-walled, clear-bottom, 96-well plates. 48 hours later, the media was aspirated and replaced with complete media and the indicated concentrations of Eos-001, trastuzumab (Tz), or a combination of trastuzumab and pertuzumab (Tz+Pz), or an untreated control at a total volume of 40 μL. Plates were rocked for 4 hours at 37° C. and 5% CO₂ and then 60 μL of complete media was added to each well to bring the total volume to 100 μL and the incubation was continued, without rocking, for 44 hours at 37° C. and 5% CO₂. At the conclusion of the incubation, relative cell viability was determined via MTS assay (Promega) according to manufacturer's instructions. Specifically, the media was removed from the wells and 100 μL of fresh complete media was added to each well. 20 μl of the prepared MTS reagent was added to each well. The plate was then incubated with rocking at 37° C. and 5% CO₂ and readings were taken of the plate at 1, 2, and 3 hours at 490 nm using a spectrophotometer. The results are shown in terms of the following ratio: number of cells that survived in the treatment group divided by the number of cells that survived in the untreated group.

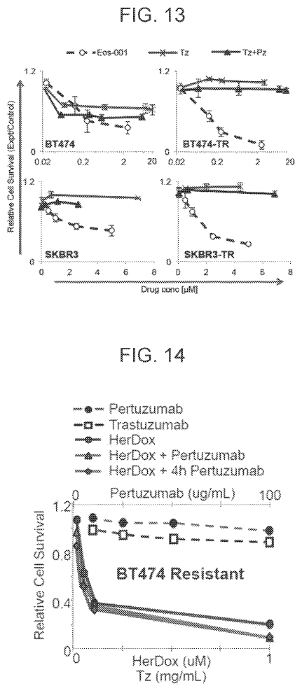

Results are shown in FIG. 13. Trastuzumab and combination trastuzumab and pertuzumab treatments were effective in killing BT474 cells, but not the BT474-TR cells. Eos-001 was effective at killing both BT474 and BT474-TR cells, demonstrating that Eos-001 nanoparticles are effective at killing cells resistant to trastuzumab and the combination of trastuzumab and pertuzumab. Neither trastuzumab nor the combination of trastuzumab and pertuzumab were effective at killing the SKBR3 or SKBR3-TR cells. The Eos-001 nanoparticles, however, were effective at killing SKBR3 and SKBR3-TR cell lines.

### Example 6: Sensitivity of BT474-TR Cells to Trastuzumab, Pertuzumab, and Eos-001 Nanoparticles

Trastuzumab or pertuzumab treatment of BT474-TR cells was compared to treatment with Eos-001, a combined treatment with Eos-001 and pertuzumab, or Eos-001 after 4 hours of pertuzumab pretreatment. The cells per well were plated in black-walled, clear-bottom, 96-well plates. 48 hours later, the media was aspirated and replaced with complete media and the indicated concentrations trastuzumab (Tz), pertuzumab (Pz), Eos-001, or the combination of Eos-001 and pertuzumab at a total volume of 40 μL. Plates were rocked for 4 hours at 37° C. and 5% CO₂ and then 60 μL of complete media was added to each well to bring the total volume to 100 μL and the incubation was continued, without rocking, for 44 hours at 37° C. and 5% CO₂. In the sample pretreated with pertuzumab before exposure to Eos-001, the cells were exposed to the indicated amount of pertuzumab for 4 hours at a total volume of 40 μL and the cells were rocked for 4 hours at 37° C. and 5% CO₂, and then 60 μL of complete media and the indicated amount of Eos-001 was added to bring the total volume to 100 μL. At the conclusion of the incubation, relative cell viability was determined via MTS assay (Promega) according to manufacturer's instructions. Specifically, the media was removed from the wells and 100 μL of fresh complete media was added to each well. 20 μl of the prepared MTS reagent was added to each well. The plate was then incubated with rocking at 37° C. and 5% CO₂ and readings were taken at 490 nm using a spectrophotometer. These results are shown in FIG. 14 and indicate that Eos-001 is more effective than trastuzumab or pertuzumab. Combining pertuzumab with Eos-001 does not result in competitive inhibition of the Eos-001 effect suggesting that the Eos-001 anticancer effect although mediated through binding to HER3 is not dependent on HER2-HER3 interaction which is disrupted by pertuzumab.

### Example 7: Eos-001 Nanoparticles Target HER3, which is Upregulated in Trastuzumab-Resistant Cells

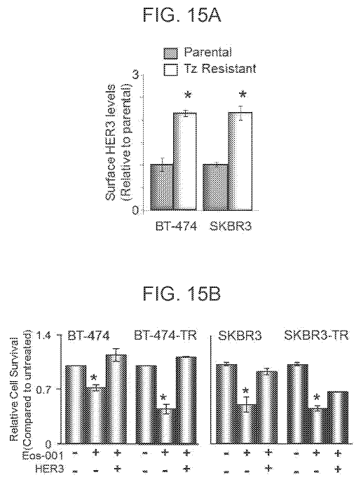

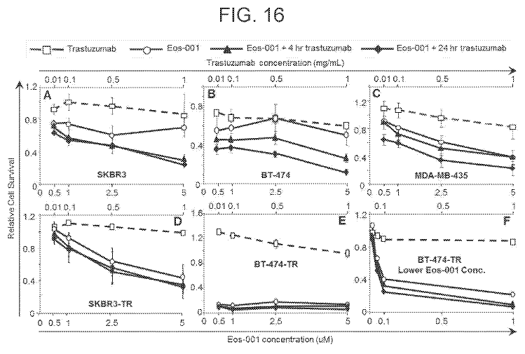

Trastuzumab-resistant BT-474-TR cells and trastuzumab-resistant SKBR3-TR cells have increased surface HER3 relative to the non-resistant parental cell lines (See FIG. 15A). To verify the contribution of HER3 targeted toxicity of the Eos-001 nanoparticles, a HER3 peptide was used as a competitive inhibitor. The HER3 peptide was pre-incubated with the Eos-001 particles, which bound the heregulin targeting domain. BT-474 cells, BT-474-TR cells, SKBR3 cells, or SKBR3-TR cells were incubated in the presence of Eos-001 nanoparticle and with or without a HER3 blocking peptide. For samples treated with Eos-001 with the HER3 blocking peptide, the nanoparticles and the HER3 blocking peptide were combined in cold PBS for one hour at an equimolar ratio of HER3:HerPBK10. The Eos-001 nanoparticles or HER3 blocking peptide treated Eos-001 nanoparticles were used to treat the cells at a final concentration of 0.125 μM (BT474 or BT474-TR cells) or 1 μM (BSKBR3 or SKBR3-TR cells). Cell survival was measured after 48 hours, and compared to cells treated with a mock saline. These results are shown in FIG. 16B (N=3, * indicates p<0.05 compared to mock). As shown in FIG. 15B, Eos-001 nanoparticles alone killed all four cell types. Surprisingly, Eos-001 was more effective at killing the BT-474-TR cells than the BT-474 cells. Presence of the HER3 peptide limited the effectiveness of Eos-001 in killing all cell types, indicating HER3 targeting of the Eos-001 particles.

### Example 8: Pre-Incubation with Trastuzumab Potentiates the Activity of Eos-001 Nanoparticles

HER3 is transcriptionally and translationally elevated in as little as 4 hours after HER2 inhibition. The enhanced efficacy of Eos-001 nanoparticles on trastuzumab-resistant cells over non-resistant cells suggests that trastuzumab may act as an adjuvant for Eos-001 nanoparticles, inducing Her3 elevation to increase targeting of Eos-001 to the resistant cells. To test this, non-trastuzumab resistant SKBR3, BT-474, and MDA-MB-435 cells, as well as trastuzumab resistant SKBR3-TR and BT-447-TR cells, were pretreated with trastuzumab for 4 or 24 hours before Eos-001 treatment. Eos-001 exhibited improved cell killing compared to trastuzumab in all cell lines, while 4 or 12 hour pre-treatment with trastuzumab resulted in increased Eos-001 potency in non-resistant cells lines. Results are shown in FIG. 16. In non-trastuzumab resistant SKBR3 cells, Eos-001 alone resulted in modest cell death at the highest dosing concentration, while a 4 or 24 hour pre-incubation with trastuzumab resulted in a 50% increase in effectivity. Similarly, non-trastuzumab resistant BT-474 cells exhibited a modest increase in cell death by Eos-001 nanoparticles over trastuzumab, with a nearly 50% increase after 4-hours pretreatment with trastuzumab, or 75% increase after 24 hours pretreatment with trastuzumab, compared to treatment with trastuzumab. Similar results were seen for MDA-MB-435 cells. In the trastuzumab-resistant cell lines (SKBR3-TR and BT474-TR), trastuzumab pre-treatment resulted in a modest increase of effectivity for Eos-001. Nevertheless, the trastuzumab-resistant SKBR3-TR and BT474-TR cell lines are effectively killed by Eos-001 without the trastuzumab pre-treatment. These results indicate that HER2 inhibitors or HER2 antibodies, such as trastuzumab, can act as a useful adjuvant for Eos-001 treatments, particularly in non-trastuzumab resistant cell lines.

### Example 9: Comparing Nanoparticles to Lapatinib Treatment in Killing Chemotherapeutic Drug Resistant Cancer Cells

BT474 (human breast cancer) cells, BT474-TR (trastuzumab-resistant human breast cancer) cells, SKBR3 (human breast cancer) cells, SKBR3-TR (trastuzumab resistant breast cancer) cells, and JIMT-1 (trastuzumab-resistant, pertuzumab-resistant human breast cancer) cells were incubated with various concentrations of Eos-001 or lapatinib. The concentration of Eos-001 is reported in μM doxorubicin, and the concentration of lapatinib is reported in μM lapatinib. The cells per well were plated in black-walled, clear-bottom, 96-well plates. 48 hours later, the media was aspirated and replaced with complete media and the indicated concentrations of Eos-001, lapatinib, or an untreated control at a total volume of 40 μL. Plates were rocked for 4 hours at 37° C. and 5% CO₂ and then 60 μL of complete media was added to each well to bring the total volume to 100 μL and the incubation was continued, without rocking, for 44 hours at 37° C. and 5% CO₂. At the conclusion of the incubation, relative cell viability was determined via MTS assay (Promega) according to manufacturer's instructions. Specifically, the media was removed from the wells and 100 μL of fresh complete media was added to each well. 20 μl of the prepared MTS reagent was added to each well. The plate was then incubated with rocking at 37° C. and 5% CO₂ and readings were taken of the plate at 1, 2, and 3 hours at 490 nm on spectrophotometer. The results are shown in terms of the following ratio: number of cells that survived in the treatment group divided by the number of cells that survived in the untreated group.

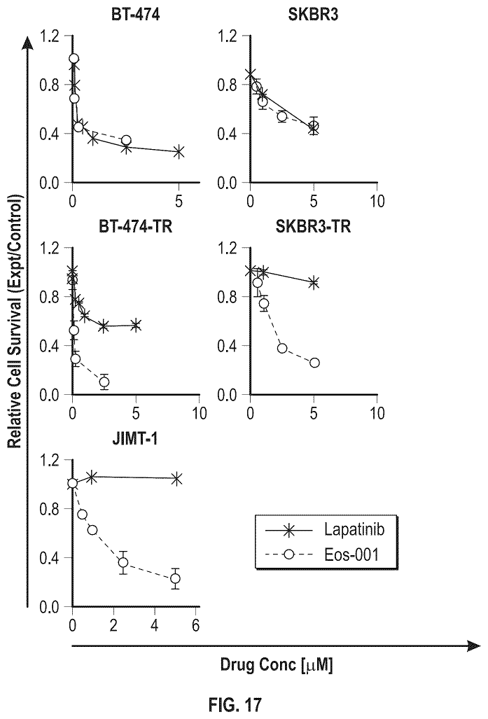

Results are shown in FIG. 17. Eos-001 (dashed line, open circles) and lapatinib (solid line) were similarly effective in treating BT-474 and SKBR3 cells. While lapatinib was slightly effective in killing trastuzumab resistant cell lines BT-474-TR and SKBR3-TR, Eos-001 was significantly more effective in killing the BT-474-TR and SKBR3-TR cell lines. Eos-001 was more effective in killing the trastuzumab resistant cell lines than the non-resistant cell lines. Further, while lapatinib was unable to kill the trastuzumab-resistant JIMT-1 cell line, Eos-001 was effective in killing these cells.

1. A composition comprising nanoparticles comprising a carrier polypeptide and a double-stranded oligonucleotide, wherein the carrier polypeptide comprises a cell-targeting segment, a cell-penetrating segment, and an oligonucleotide-binding segment; and
wherein the molar ratio of the carrier polypeptide to the double-stranded oligonucleotide in the nanoparticle composition is less than about 6:1.

2-35. (canceled)

36. A method of treating a subject with a chemotherapeutic drug-resistant cancer, comprising administering to the subject a composition comprising nanoparticles, the nanoparticles comprising:
a carrier polypeptide comprising a cell-targeting segment, a cell-penetrating segment, and an oligonucleotide-binding segment;
a double-stranded oligonucleotide bound to the oligonucleotide-binding segment; and
a chemotherapeutic drug bound to the double-stranded oligonucleotide;
wherein the chemotherapeutic drug-resistant cancer is an anthracycline-resistant cancer or a taxane-resistance cancer.

37-48. (canceled)

49. A method of making a nanoparticle composition comprising:
combining a carrier polypeptide and a double-stranded oligonucleotide at a molar ratio of less than about 6:1, thereby forming a plurality of nanoparticles;
wherein the carrier polypeptide comprises a cell-targeting segment, a cell-penetrating segment, and an oligonucleotide-binding segment.

50. The method of claim 49, wherein the molar ratio of the carrier polypeptide to the double-stranded oligonucleotide is about 4:1 to less than about 6:1.

51. The method of claim 49, wherein the molar ratio of the carrier polypeptide to the double-stranded oligonucleotide is about 4:1.

52. The method of claim 49, further comprising combining the double-stranded oligonucleotide and a small-molecule drug prior to combining the double-stranded oligonucleotide and the carrier polypeptide.

53. The method of claim 52, wherein the double-stranded oligonucleotide and the small-molecule drug are combined at a molar ratio of about 1:1 to about 1:60.

54. The method of claim 52, wherein the double-stranded oligonucleotide and the small-molecule drug are combined at a molar ratio of about 1:10 or about 1:40.

55. The method of claim 52, further comprising separating unbound small-molecule drug from the double-stranded oligonucleotide prior to combining the double-stranded oligonucleotide and the carrier polypeptide.

56. The method of claim 49, further comprising separating unbound carrier polypeptide or unbound double-stranded oligonucleotide from the plurality of nanoparticles.

57. The method of claim 49, further comprising concentrating the nanoparticle composition.

58. The method of claim 49, wherein the double-stranded oligonucleotide is DNA.

59. The method of claim 49, wherein the double-stranded oligonucleotide is RNA.

60. The method of claim 49, wherein the double-stranded oligonucleotide is about 10 base pairs to about 100 base pairs in length.

61. The method of claim 49, wherein the small-molecule drug is a chemotherapeutic agent.

62. The method of claim 49, wherein the small-molecule drug is an anthracycline or a taxane.

63. The method of claim 49, wherein the small-molecule drug is doxorubicin.

64. The method of claim 49, wherein the cell-targeting segment comprises a heregulin sequence or a variant thereof.

65. The method of claim 49, wherein the cell-penetrating segment comprises a penton base polypeptide or a variant thereof.

66. (canceled)

67. (canceled)

68. The method of claim 49, wherein the oligonucleotide-binding segment is positively charged.

69-71. (canceled)
